(19) United States
(12) Patent Application Publication
(10) Pub. No.: US 2026/0335347 A1
(43) Pub. Date: Oct. 29, 2026

(54) COMBINATION THERAPY WITH SPARSENTAN AND A SGLT2 INHIBITOR FOR TREATING KIDNEY DISEASES OR DISORDERS

(71) Applicant: Travere Therapeutics, Inc., San Diego, CA (US)

(72) Inventor: Radko Komers, Portland, OR (US)

(21) Appl. No.: 19/116,148

(22) PCT Filed: Sep. 29, 2023
(86) PCT No.: PCT/US2023/075525

§ 371 Date: Mar. 27, 2025

Related U.S. Application Data
(60) Provisional application No. 63/412,252, filed on Sep. 30, 2022.

(51) Int. Cl. A61K 31/7048; A61K 9/00; A61K 9/20; A61K 31/382; A61K 31/422; A61K 31/7004; A61P 13/12

(52) U.S. Cl. CPC A61K 31/7048; A61K 9/0053; A61K 9/2009; A61K 9/2018; A61K 9/2054; A61K 9/2059; A61K 31/382; A61K 31/422; A61K 31/7004; A61P 13/12

(57) ABSTRACT

The present disclosure relates to methods of treating a kidney disease or disorder, including methods comprising administering sparsentan and a SGLT2 inhibitor to a subject in need thereof.

### BACKGROUND

The present disclosure relates to the use of sparsentan, a dual angiotensin and endothelin receptor antagonist, and a sodium-glucose cotransporter-2 (SGLT2) inhibitor in the treatment of kidney diseases or disorders, such as focal segmental glomerulosclerosis (FSGS) and immunoglobulin A nephropathy (IgAN).

FSGS is a rare disease that affects the kidneys. Patients with FSGS exhibit scarring of the glomeruli of the kidney. Glomeruli filter the blood and remove water and some toxins, producing urine and leaving proteins behind in the blood. The scarring of the glomeruli in patients with FSGS is associated with leakage of protein into the urine (instead of remaining in the blood), a condition called proteinuria. Proteinuria causes fluid to build up in the body, and protracted proteinuria may itself result in damage to the kidneys and kidney dysfunction. FSGS is categorized as primary (or “idiopathic”), secondary, or genetic. Primary FSGS has no known etiology. Secondary FSGS may be caused by reduction in renal mass, including that which may be associated with low birth weight; vesicoureteral reflux; obesity; medications; infections, including HIV infection; or systemic illnesses, such as diabetes, sickle cell anemia, and lupus. If FSGS goes untreated, it can lead to end-stage renal disease (ESRD) over five to ten years.

IgAN, also known as Berger's disease, is caused by the buildup of immunoglobulin A (IgA) in the kidney. The presence of IgA in the kidneys may lead to inflammation, damage to the glomeruli of the kidney, and impaired kidney function, including proteinuria. In some cases, patients with IgA nephropathy progress to end-stage renal disease (ESRD).

Angiotensin II (AngII) and endothelin-I (ET-1) are two of the most potent endogenous vasoactive peptides currently known and are believed to play a role in controlling both vascular tone and pathological tissue remodeling associated with a variety of diseases including diabetic nephropathy, heart failure, and chronic or persistently elevated blood pressure. Angiotensin receptor blockers (ARBs), which block the activity of AngII, have been used as a treatment for diabetic nephropathy, heart failure, chronic, or persistently elevated blood pressure. There is also a growing body of data that demonstrates the potential therapeutic benefits of ET receptor antagonists (ERAs) in blocking ET-1 activity. Additionally, AngII and ET-1 are believed to work together in blood pressure control and pathological tissue remodeling. For example, ARBs not only block the action of AngII at its receptor, but also limit the production of ET-1. Similarly, ERAs block ET-1 activity and inhibit the production of AngII. Consequently, simultaneously blocking AngII and ET-1 activities may offer better efficacy than blocking either substance alone.

Reduction in proteinuria is associated with lower risk of end-stage renal disease (ESRD) in patients having FSGS (Troost et al., Clin J Am Soc Nephrol 13:414-421, 2018; International Patent Application Publication No. WO2018/071784 A1). In a Phase 2 clinical study, sparsentan was shown to reduce proteinuria in patients with FSGS (International Patent Application Publication No. WO2018/071784 A1). Sparsentan was approved by United States Food and Drug Administration for the reduction of proteinuria in adults with IgA nephropathy at high risk of disease progression, based on the PROTECT clinical trial (ClinicalTrials.gov, NCT03762850) (Heerspink et al., Lancet 401 (10388): 1584-1594, 2023 (published Apr. 1, 2023), doi: 10.1016/S0140-6736 (23) 00569-X).

SGLT2 inhibitors are a class of molecules that are FDA-approved for the treatment of type 2 diabetes (http://www.fda.gov/drugs/postmarket-drug-safety-information-patients-and-providers/sodium-glucose-cotransporter-2-sglt2-inhibitors). They include canagliflozin, dapagliflozin (e.g., FARXIGA®), and empagliflozin (e.g., JARDIANCE®), among others. SGLT2 inhibitors have been suggested as a therapeutic option for slowing disease progression in patients with diabetic and nondiabetic chronic kidney disease (Mende, Adv Ther (2022) 39:148-164). Use of a SGLT2 inhibitor and a selective endothelin A receptor antagonist in a combination therapy for treating IgAN has been described (International Patent Application Publication No. WO2021126977A1).

There remains a need for treatments of kidney diseases or disorders, including those associated with elevated proteinuria.

### BRIEF SUMMARY

The present disclosure provides, in some embodiments, methods of treating a kidney disease or disorder, wherein the method comprises administering a compound having structure (I),

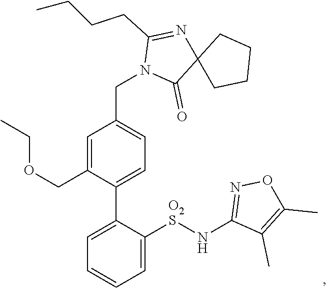 [SMILES: CCCCC1=NC2(CCCC2)C(=O)N1Cc1ccc(-c2ccccc2S(=O)(=O)Nc2noc(C)c2C)c(COCC)c1]

or a pharmaceutically acceptable salt thereof, and a SGLT2 inhibitor, to a subject (e.g., a human patient) in need thereof. In some embodiments, the kidney disease or disorder is a proteinuric disease, such as focal segmental glomerulosclerosis (FSGS) or immunoglobulin A nephropathy (IgAN).

These and other aspects of the present invention will become apparent upon reference to the following detailed description.

### DETAILED DESCRIPTION

The present disclosure generally relates to the use of biphenyl sulfonamide compounds that have dual angiotensin and endothelin receptor antagonist activity, such as sparsentan, in combination with a SGLT2 inhibitor, in the treatment of kidney diseases or disorders, e.g., proteinuric diseases, including focal segmental glomerulosclerosis (FSGS) and immunoglobulin A nephropathy (IgAN).

In the following description, certain specific details are set forth in order to provide a thorough understanding of various embodiments of the invention. However, one skilled in the art will understand that the invention may be practiced without these details.

Unless defined otherwise, all technical and scientific terms used herein have the same meaning as is commonly understood by one of skill in the art to which this invention belongs. As used herein, certain terms may have the following defined meanings.

Unless the context requires otherwise, throughout the present specification and claims, the word “comprise” and variations thereof, such as “comprises” and “comprising,” are to be construed in an open, inclusive sense, that is, as “including, but not limited to.”

As used in the specification and claims, “including” and variants thereof, such as “include” and “includes,” are to be construed in an open, inclusive sense; i.e., it is equivalent to “including, but not limited to.” As used herein, the terms “include” and “have” are used synonymously, which terms and variants thereof are intended to be construed as non-limiting.

As used in herein, the phrase “such as” refers to non-limiting examples.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in the specification and claims, the singular for “a,” “an,” and “the” include plural references unless the context clearly dictates otherwise. For example, the term “a cell” includes a plurality of cells, including mixtures thereof. Similarly, use of “a compound” for treatment of preparation of medicaments as described herein contemplates using one or more compounds of the invention for such treatment or preparation unless the context clearly dictates otherwise.

The use of the alternative (e.g., “or”) should be understood to mean either one, both, or any combination thereof of the alternatives.

“Optional” or “optionally” means that the subsequently described event of circumstances may or may not occur, and that the description includes instances where said event or circumstance occurs and instances in which it does not occur.

As used herein, “about” and “approximately” generally refer to an acceptable degree of error for the quantity measured, given the nature or precision of the measurements. Typical, exemplary degrees of error may be within 20%, 10%, or 5% of a given value or range of values. Alternatively, and particularly in biological systems, the terms “about” and “approximately” may mean values that are within an order of magnitude, potentially within 5-fold or 2-fold of a given value. When not explicitly stated, the terms “about” and “approximately” mean equal to a value, or within 20% of that value.

As used herein, numerical quantities are precise to the degree reflected in the number of significant figures reported. For example, a value of 0.1 is understood to mean from 0.05 to 0.14. As another example, the interval of values 0.1 to 0.2 includes the range from 0.05 to 0.24.

The compound having structure (I) may form salts, which are also within the scope of this disclosure. Reference to a compound having structure (I) herein is generally understood to include reference to salts thereof, unless otherwise indicated. The term “salt(s),” as employed herein, denotes acidic, or basic salts formed with inorganic or organic acids and bases. In addition, as the compound having structure (I) contains both a basic moiety and an acidic moiety, zwitterions (“inner salts”) may be formed and are included within the term “salt(s),” as used herein. Pharmaceutically acceptable (i.e., non-toxic, physiologically acceptable) salts are preferred, although other salts may be useful, e.g., in isolation or purification steps which may be employed during preparation. Salts of the compound having structure (I) may be formed, for example, by reacting the compound having structure (I) with an amount of acid or base, such as an equivalent amount, in a medium such as one in which the salt precipitates or in an aqueous medium followed by lyophilization.

The term “pharmaceutically acceptable salt” includes both acid and base addition salts.

Prodrugs and solvates of the compound having structure (I) are also contemplated. The term “prodrug” denotes a compound that, upon administration to a subject, undergoes chemical conversion by metabolic or chemical processes to yield a compound having structure (I), or a salt or solvate thereof. Solvates of the compound having structure (I) may be hydrates. Any tautomers are also contemplated.

Crystallizations may produce a solvate of the compound having structure (I), or a salt thereof. As used herein, the term “solvate” refers to an aggregate that comprises one or more molecules of a compound as disclosed herein with one or more molecules of solvent. In some embodiments, the solvent is water, in which case the solvate is a hydrate. Alternatively, in other embodiments, the solvent is an organic solvent. Thus, the compounds of the present disclosure may exist as a hydrate, including a monohydrate, dihydrate, hemihydrate, sesquihydrate, trihydrate, tetrahydrate, and the like, as well as the corresponding solvated forms. In some embodiments, the compounds disclosed herein may be a true solvate, while in other cases, the compounds disclosed herein merely retain adventitious water or are mixtures of water plus some adventitious solvent.

The invention disclosed herein is also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, the invention includes compounds produced by a process comprising administering a compound of this invention to a mammal for a period of time sufficient to yield a metabolic product thereof. Such products are typically identified by administering a radiolabeled compound of the invention in a detectable dose to an animal, such as rat, mouse, guinea pig, monkey, or to human, allowing sufficient time for metabolism to occur, and isolating its conversion products from the urine, blood, or other biological samples.

“Stable compound” and “stable structure” are meant to indicate a compound that is sufficiently robust to survive isolation to a useful degree of purity from a reaction mixture, and formulation into an efficacious therapeutic agent.

The term “subject” refers to a mammal, such as a domestic pet (for example, a dog or cat), or human. Preferably, the subject is a human. In some embodiments, the subject is a patient that has been diagnosed as having a disease or disorder (e.g., IgAN or FSGS).

The phrase “effective amount” refers to the amount which, when administered to a subject or patient for treating a disease, is sufficient to effect such treatment for the disease.

The term “unit dosage form” (or “unit dose form”) is the form of a pharmaceutical product, including, but not limited to, the form in which the pharmaceutical product is marketed for use. Examples include pills, tablets, capsules, and liquid solutions and suspensions.

“Treatment” or “treating” includes (1) inhibiting a disease in a subject or patient experiencing or displaying the pathology or symptomatology of the disease (e.g., arresting further development of the pathology or symptomatology); or (2) ameliorating a disease in a subject or patient that is experiencing or displaying the pathology or symptomatology of the disease (e.g., reversing the pathology or symptomatology); or (3) effecting any measurable decrease in a disease in a subject or patient that is experiencing or displaying the pathology or symptomatology of the disease.

“Preventing” in the context of preventing a subject (e.g., a patient) from experiencing or displaying the pathology or symptomology of a disease includes the failure to develop a disease, disorder, or condition, or the reduction in the development of a sign or symptom associated with such a disease, disorder, or condition (e.g., by a clinically relevant amount), or the exhibition of delayed signs or symptoms delayed (e.g., by days, weeks, months, or years).

Additional definitions are set forth throughout this disclosure.

### Sparsentan

Sparsentan (CAS 254740-64-2, 2-[4-[(2-butyl-4-oxo-1,3-diazaspiro[4.4]non-1-en-3-yl)methyl]-2-(ethoxymethyl)phenyl]-N-(4,5-dimethyl-1,2-oxazol-3-yl)benzenesulfonamide) is a biphenyl sulfonamide compound having structure (I),

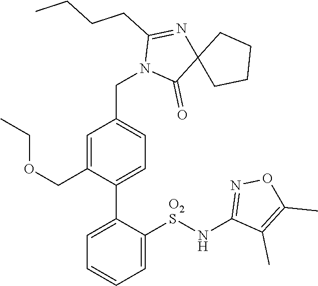 [SMILES: CCCCC1=NC2(CCCC2)C(=O)N1Cc1ccc(-c2ccccc2S(=O)(=O)Nc2noc(C)c2C)c(COCC)c1]

Sparsentan is a selective dual-acting receptor antagonist with affinity for endothelin (A type) receptors (“ET_{A}” receptors) and angiotensin II receptors (Type 1) (“AT₁” receptors) (Kowala et al., JPET 309:275-284, 2004).

The compound of structure (I) may be prepared by methods such as those described in International Patent Application Publication No. WO2018/071784 A1 and U.S. Patent Application Publication No. US 2019/0262317 A1, which references are incorporated herein by reference in their entireties. Additionally, the compound of structure (I) may be prepared by the methods recited in U.S. Patent Application Publication No. US 2015/0164865 A1 and U.S. Pat. No. 6,638,937 B2, which references are incorporated herein by reference in their entireties.

### Sodium-Glucose Cotransporter-2 (SGLT2) Inhibitors

SGLT2 inhibitors are a class of molecules that have been shown to result in significant improvement in kidney function in diabetic patients suffering from diabetic nephropathy, as well as protection of kidneys in patients with chronic kidney disease who do not have diabetes (https://www.kidney.org/atoz/content/sglt2-inhibitors, accessed Sep. 26, 2022; Mende, Adv Ther 39:148-164, 2022). SGLT2 inhibitors have been approved by the United States Food and Drug Administration for the treatment of type 2 diabetes (http://www.fda.gov/drugs/postmarket-drug-safety-information-patients-and-providers/sodium-glucose-cotransporter-2-sglt2-inhibitors, accessed Sep. 26, 2022).

As used herein, the term “SGLT2 inhibitor” refers to a substance (e.g., a compound or molecule) that inhibits activity or expression of SGLT2. SGLT2 inhibitors include compounds that inhibit SGLT2 activity, for example, gliflozins, such as canagliflozin (e.g., INVOKANA®, TA-7284), dapagliflozin (e.g., FARXIGA®), empagliflozin (e.g., JARDIANCE®, BI 10773), ertugliflozin (e.g., STEGLAGTRO®), ipragliflozin (e.g., SUGLAT®, ASP-1941), tofogliflozin or tofogliflozin hydrate (e.g., DEBERZA®, APLEWAY®, R-7201), luseogliflozin (e.g., LUSEFI®, TS-071), sotagliflozin (e.g., ZYNQUISTA™, LX4211), remogliflozin and remogliflozin etabonate (GSK189075), bexagliflozin (EGT0001442), sergliflozin and sergliflozin etabonate, licogliflozin (LIK 066), janagliflozin (XZP-5695), henagliflozin (SHR-3824), and enavogliflozin (DWP-16001); BI 44847; EGT0001474; YM-543 (ASP-543); TS-033; TA-1887 (3-(4-cyclopropylbenzyl)-4-fluoroindole-N-glucoside 6a-4); indole-N-glycoside 18 (3-(4-ethylbenzyl)-1-(β-D-glucopyranosyl)-1H-indole); and T-1095 (((2R,3S,4S,5R,6S)-6-(2-(3-(benzofuran-5-yl) propanoyl)-3-hydroxy-5-methylphenoxy)-3,4,5-trihydroxytetrahydro-2H-pyran-2-yl) etabonate); as well as pharmaceutically acceptable salts, solvates, complexes, and salts of solvates thereof. SGLT2 inhibitors also include molecules that modulate SGLT2 activity by inhibiting expression of SGLT2 proteins, for example, siRNA molecules; and antisense molecules, such as ISIS-SGLT2Rx. See, e.g., Oku et al., Diabetes 48 (9): 1794-1800, 1999; Isaji et al., Kidney Int 79 (Suppl 120): S14-S19, 2011; Nomura et al., ACS Med Chem Lett. 5 (1): 51-55, 2013; Haas et al., Nutr & Diabetes 4: e143, 2014; Hsia et al., Curr Opin Endocrinol Diabetes Obes. 24 (1): 73-79, 2017; Bhatt et al., N Engl J Med 384:117-128, 2021; Dobbins et al., BMC Pharmacol Toxicol 22:34, 2021.

### Pharmaceutical Compositions

In some embodiments, the present disclosure relates to the administration of (i) a pharmaceutical composition comprising a compound of structure (I), or pharmaceutically acceptable salt thereof, and (ii) a pharmaceutical composition comprising a SGLT2 inhibitor. In some embodiments, the compound of structure (I), or pharmaceutically acceptable salt thereof, and the SGLT2 inhibitor are formulated together in the same pharmaceutical composition.

The term “pharmaceutical composition” as used herein refers to a composition comprising an active ingredient and a pharmaceutically acceptable excipient. Pharmaceutical compositions may be used to facilitate administration of an active ingredient to an organism. Multiple techniques of administering a compound exist in the art, such as oral, injection, aerosol, parenteral, and topical administration. Pharmaceutical compositions can be obtained, for example, by reacting compounds with inorganic or organic acids such as hydrochloric acid, hydrobromic acid, sulfuric acid, nitric acid, phosphoric acid, methane sulfonic acid, ethanesulfonic acid, p-toluenesulfonic acid, salicylic acid, and the like. As used herein, the term “physiologically acceptable excipient” or “pharmaceutically acceptable excipient” refers to a physiologically and pharmaceutically suitable non-toxic and inactive material or ingredient that does not interfere with the activity of the active ingredient, including any adjuvant, carrier, glidant, sweetening agent, diluent, preservative, dye/colorant, flavor enhancer, surfactant, wetting agent, dispersing agent, suspending agent, stabilizer, isotonic agent, solvent, or emulsifier that has been approved by the United States Food and Drug Administration as being acceptable for use in humans or domestic animals.

In some embodiments, the pharmaceutical composition may be formulated as described in further detail below.

Also provided herein are kits including components of a combination therapy. In some embodiments, the kit comprises: a pharmaceutical composition comprising sparsentan; and a pharmaceutical composition comprising a SGLT2 inhibitor. In some embodiments, the kit is for use in any one of the methods described herein.

### Methods of Treatment and Other Uses

Additionally, methods of treating a kidney disease or disorder comprising administering a combination therapy are within the scope of the present disclosure, wherein the combination therapy comprises administering (i) a compound of structure (I), or pharmaceutically acceptable salt thereof, e.g., sparsentan, and (ii) a SGLT2 inhibitor, to a subject in need thereof. In some embodiments, the present disclosure provides methods of treating a kidney disease or disorder comprising administering a combination therapy, wherein the combination therapy comprises administering (i) a pharmaceutical composition comprising a compound of structure (I), or pharmaceutically acceptable salt thereof, e.g., sparsentan, and a pharmaceutically acceptable excipient, and (ii) a pharmaceutical composition comprising a SGLT2 inhibitor, and a pharmaceutically acceptable excipient, to a subject in need thereof.

In some embodiments, a combination therapy as described above is useful in the treatment of a kidney disease or disorder. In some embodiments, a combination therapy as described above is useful in the treatment of a proteinuric disease. In some embodiments, a combination therapy as described above is useful in the reduction of general morbidity or mortality as a result of such utilities.

In some embodiments, a combination therapy as described above is useful in prolonging the time to end stage renal disease or reducing a risk of end stage renal disease in a subject, e.g., a patient diagnosed with FSGS or IgAN.

In some embodiments, a combination therapy as described above is useful in reducing proteinuria. As used herein, “proteinuria” refers to a condition in which the urine contains an abnormal amount of protein (i.e., urine protein excretion of greater than 300 mg per day). A urinary protein to creatinine (“UP/C”) ratio provides a measurement of total urine protein relative to the amount of creatinine in a urine sample (e.g., 1 g of protein in urine (dl) divided by 1 g of creatinine in urine (dl)=a UP/C ratio of 1). As used herein, a UP/C ratio of more than 0.3 g/g indicates proteinuria. In some embodiments, the combination therapy is useful in reducing proteinuria to less than or equal to 1.5 g/g. In some embodiments, the combination therapy is useful in reducing proteinuria to less than or equal to 1.0 g/g. In some embodiments, the combination therapy is useful in reducing proteinuria to less than or equal to 0.5 g/g. In some embodiments, the combination therapy is useful in reducing proteinuria to less than or equal to 0.3 g/g (i.e., complete remission of proteinuria). Accordingly, the present disclosure also provides methods of inducing complete remission in a subject having a kidney disease or disorder, e.g., FSGS or IgAN, by administering the combination therapy to the subject.

In any of the aforementioned embodiments, the method may achieve a reduction in the subject's UP/C ratio of at least 40% relative to the subject's baseline UP/C ratio. As used herein, a subject's “baseline UP/C ratio” refers to the subject's most recently calculated UP/C ratio prior to onset of treatment.

In some embodiments, the dosing regimen comprises administering the compound having structure (I), or pharmaceutically acceptable salt thereof, in an amount of 100 mg/day. In some embodiments, the dosing regimen comprises administering the compound having structure (I), or pharmaceutically acceptable salt thereof, in an amount of 200 mg/day. In some embodiments, the dosing regimen comprises administering the compound having structure (I), or pharmaceutically acceptable salt thereof, in an amount of 400 mg/day. In some embodiments, the dosing regimen comprises administering the compound having structure (I), or pharmaceutically acceptable salt thereof, in an amount of 800 mg/day. In some embodiments, the dosing regimen comprises administering the compound having structure (I), or pharmaceutically acceptable salt thereof, in an amount of 200 mg/day for 8 weeks, 26 weeks, 36 weeks, 8 months, or 108 weeks. In some embodiments, the dosing regimen comprises administering the compound having structure (I), or pharmaceutically acceptable salt thereof, in an amount of 400 mg/day for 8 weeks, 26 weeks, 36 weeks, 8 months, or 108 weeks. In some embodiments, the dosing regimen comprises administering the compound having structure (I), or pharmaceutically acceptable salt thereof, in an amount of 800 mg/day for 8 weeks, 26 weeks, 36 weeks, 8 months, or 108 weeks. In some embodiments, the dosing regimen comprises administering the compound having structure (I), or pharmaceutically acceptable salt thereof, in an amount of 200 mg/day for 6 weeks, 36 weeks, 58 weeks, or 110 weeks. In some embodiments, the dosing regimen comprises administering the compound having structure (I), or pharmaceutically acceptable salt thereof, in an amount of 400 mg/day for 6 weeks, 36 weeks, 58 weeks, or 110 weeks.

In some embodiments, if the subject's weight is from 20 kg to 50 kg, the amount of the compound having structure (I), or pharmaceutically acceptable salt thereof, administered to the subject is 200 mg/day for the first 2 weeks and thereafter is 400 mg/day. In some embodiments, if the subject's weight is greater than 50 kg, the amount of the compound having structure (I), or pharmaceutically acceptable salt thereof, administered to the subject is 400 mg/day for the first 2 weeks and thereafter is 800 mg/day.

In some embodiments, a method of treating a kidney disease or disorder in a subject in need thereof is provided, the method comprising administering to the subject, over an administration period, the combination therapy in an amount sufficient to achieve or maintain a UP/C ratio of less than or equal to 1.5 g/g for at least a portion of the administration period. In some embodiments, a method of treating a kidney disease or disorder in a subject in need thereof is provided, the method comprising administering to the subject, over an administration period, the combination therapy in an amount sufficient to achieve or maintain a UP/C ratio of less than or equal to 1.0 g/g for at least a portion of the administration period. “Administration period” refers to the time period during which the pharmaceutical composition is administered to the subject as least daily. In some embodiments, the administration period is 6 weeks. In some embodiments, the administration period is 8 weeks. In some embodiments, the administration period is 26 weeks. In some embodiments, the administration period is 36 weeks. In some embodiments, the administration period is 108 weeks. In some embodiments, the administration period is 110 weeks. In some embodiments, the administration period is 8 months.

In some embodiments, a method of maintaining a UP/C ratio at less than or equal to 1.5 g/g in a subject in need thereof is provided, the method comprising administering to the subject the combination therapy in an amount sufficient to maintain a UP/C ratio of less than or equal to 1.5 g/g.

In some embodiments, a method of maintaining a UP/C ratio at less than or equal to 1.0 g/g in a subject in need thereof is provided, the method comprising administering to the subject the combination therapy in an amount sufficient to maintain a UP/C ratio of less than or equal to 1.0 g/g.

In some embodiments, a method of reducing a UP/C ratio to less than or equal to 1.5 g/g in a subject in need thereof is provided, comprising administering to the subject the combination therapy in an amount sufficient to reduce said subject's UP/C ratio to less than or equal to 1.5 g/g. In some embodiments, the subject has, or has had, a UP/C ratio greater than 1.5 g/g prior to administration of the combination therapy.

In some embodiments, a method of reducing a UP/C ratio to less than or equal to 1.0 g/g in a subject in need thereof is provided, comprising administering to the subject the combination therapy in an amount sufficient to reduce said subject's UP/C ratio to less than or equal to 1.0 g/g. In some embodiments, the subject has, or has had, a UP/C ratio greater than 1.0 g/g prior to administration of the combination therapy.

In some embodiments, the combination therapy is useful in maintaining glomerular filtration rate. As used herein, “glomerular filtration rate” (“GFR”) is a measure of kidney function and refers to the amount of fluid filtered through the glomeruli of the kidney per unit of time. GFR may be estimated by measuring serum creatinine levels and using the Chronic Kidney Disease Epidemiology Collaboration (CKD-EPI) creatinine equation. As used herein, “estimated glomerular filtration rate” (“eGFR”) refers to an estimate of GFR obtained from using the CKD-EPI creatinine equation. In some embodiments, the combination therapy is useful in maintaining eGFR levels (e.g., preventing a reduction in eGFR associated with a kidney disease or disorder, such as FSGS or IgAN, or reducing the rate of decline in eGFR in a subject having a kidney disease or disorder, such as FSGS or IgAN). In some embodiments, administering the combination therapy to a subject results in eGFR being maintained at or above eGFR levels immediately prior to (e.g., within a month prior to) administration of the combination therapy. In some embodiments, administering the combination therapy to a subject results in eGFR being maintained at or above baseline eGFR level, where “baseline eGFR level” refers to their most recently calculated eGFR level prior to onset of treatment. As used herein, “maintenance of eGFR” refers to no clinically meaningful reduction in baseline eGFR levels. Thus, as used herein, in reference to treatment of a subject (e.g., a patient), the phrase “maintain eGFR constant” means treatment that maintains the subject's eGFR at a level that is clinically equivalent to or better than their baseline eGFR (i.e., most recently calculated eGFR level prior to onset of treatment). In some embodiments, the eGFR is maintained for months or years after administration. The period of time during which the subject's eGFR level is maintained constant typically is at least 12 months.

In some embodiments of the aforementioned uses or methods of treatment, treatment with the combination therapy is more effective than treatment with the compound of structure (I), or pharmaceutically acceptable salt thereof, e.g., sparsentan, or the SGLT2 inhibitor alone. In some embodiments, the combination therapy prolongs the time to end stage renal disease or reduces a risk of end stage renal disease in a subject, e.g., a patient diagnosed with FSGS or IgAN, relative to treatment with the compound of structure (I), or pharmaceutically acceptable salt thereof, e.g., sparsentan, or the SGLT2 inhibitor alone. In some embodiments, the combination therapy provides a greater reduction in proteinuria in a subject, e.g., a patient diagnosed with FSGS or IgAN, relative to treatment with the compound of structure (I), or pharmaceutically acceptable salt thereof, e.g., sparsentan, or the SGLT2 inhibitor alone. In some embodiments, the combination therapy results in complete remission of proteinuria (reduced proteinuria to less than or equal to 0.3 g/g) in a subject, e.g., a patient diagnosed with FSGS or IgAN, more than treatment with the compound of structure (I), or pharmaceutically acceptable salt thereof, e.g., sparsentan, or the SGLT2 inhibitor alone. In some embodiments, the combination therapy achieves a reduction in the subject's UP/C ratio of at least 40% relative to the subject's baseline UP/C ratio in a subject, e.g., a patient diagnosed with FSGS or IgAN, more than treatment with the compound of structure (I), or pharmaceutically acceptable salt thereof, e.g., sparsentan, or the SGLT2 inhibitor alone. In some embodiments, the combination maintains eGFR levels in a subject, e.g., a patient diagnosed with FSGS or IgAN, more than treatment with the compound of structure (I), or pharmaceutically acceptable salt thereof, e.g., sparsentan, or the SGLT2 inhibitor alone.

In some embodiments of the aforementioned uses or methods of treatment, the use or method comprises administering the combination therapy as a fixed dose combination formulation.

In some embodiments, any of the aforementioned uses or methods of treatment may comprise administering the combination therapy in combination with one or more other active ingredients, such as other therapeutic or diagnostic agents. For example, in some embodiments, one or more other therapeutic agents may be administered prior to, simultaneously with, or following the administration of the combination therapy. If formulated as a fixed dose, such combination products may employ the combination therapy, within the dosage ranges described herein, and the other active ingredient within its approved dosage range.

In some embodiments, the combination therapy is used in conjunction with hemodialysis.

In some embodiments of the aforementioned uses and methods of treatment, the dosing regimen comprises administering the compound having structure (I), or pharmaceutically acceptable salt thereof, in an amount of 50 mg/day. In some embodiments, the dosing regimen comprises administering the compound having structure (I), or pharmaceutically acceptable salt thereof, in an amount of 100 mg/day. In some embodiments, the dosing regimen comprises administering the compound having structure (I), or pharmaceutically acceptable salt thereof, in an amount of 200 mg/day. In some embodiments, the dosing regimen comprises administering the compound having structure (I), or pharmaceutically acceptable salt thereof, in an amount of 300 mg/day. In some embodiments, the dosing regimen comprises administering the compound having structure (I), or pharmaceutically acceptable salt thereof, in an amount of 400 mg/day. In some embodiments, the dosing regimen comprises administering the compound having structure (I), or pharmaceutically acceptable salt thereof, in an amount of 500 mg/day. In some embodiments, the dosing regimen comprises administering the compound having structure (I), or pharmaceutically acceptable salt thereof, in an amount of 600 mg/day. In some embodiments, the dosing regimen comprises administering the compound having structure (I), or pharmaceutically acceptable salt thereof, in an amount of 700 mg/day. In some embodiments, the dosing regimen comprises administering the compound having structure (I), or pharmaceutically acceptable salt thereof, in an amount of 800 mg/day. In some embodiments, the dosing regimen comprises administering the compound having structure (I), or pharmaceutically acceptable salt thereof, in an amount of 900 mg/day. In some embodiments, the dosing regimen comprises administering the compound having structure (I), or pharmaceutically acceptable salt thereof, in an amount of 1000 mg/day.

In some embodiments of the aforementioned uses and methods of treatment, the dosing regimen comprises administering the compound having structure (I), or pharmaceutically acceptable salt thereof, in an amount of 100 mg/day for 6 weeks, 8 weeks, 26 weeks, 36 weeks, 8 months, 108 weeks, or 110 weeks. In some embodiments of the aforementioned uses and methods of treatment, the dosing regimen comprises administering the compound having structure (I), or pharmaceutically acceptable salt thereof, in an amount of 200 mg/day for 6 weeks, 8 weeks, 26 weeks, 36 weeks, 8 months, 108 weeks, or 110 weeks. In some embodiments of the aforementioned uses and methods of treatment, the dosing regimen comprises administering the compound having structure (I), or pharmaceutically acceptable salt thereof, in an amount of 400 mg/day for 6 weeks, 8 weeks, 26 weeks, 36 weeks, 8 months, 108 weeks, or 110 weeks. In still further embodiments, the dosing regimen comprises administering the compound having structure (I), or pharmaceutically acceptable salt thereof, in an amount of 800 mg/day for 6 week, 8 weeks, 26 weeks, 36 weeks, 8 months, 108 weeks, or 110 weeks.

In any of the aforementioned embodiments, the amount of the compound having structure (I), or pharmaceutically acceptable salt thereof, administered to the subject may be from about 50 mg/day to about 1000 mg/day. For example, in some embodiments, the amount of the compound having structure (I), or pharmaceutically acceptable salt thereof, administered to the subject is from about 200 mg/day to about 800 mg/day. In other embodiments, the amount of the compound having structure (I), or pharmaceutically acceptable salt thereof, administered to the subject is about 50 mg/day. In other embodiments, the amount of the compound having structure (I), or pharmaceutically acceptable salt thereof, administered to the subject is about 100 mg/day. In other embodiments, the amount of the compound having structure (I), or pharmaceutically acceptable salt thereof, administered to the subject is about 200 mg/day. In other embodiments, the amount of the compound having structure (I), or pharmaceutically acceptable salt thereof, administered to the subject is about 300 mg/day. In other embodiments, the amount of the compound having structure (I), or pharmaceutically acceptable salt thereof, administered to the subject is about 400 mg/day. In other embodiments, the amount of the compound having structure (I), or pharmaceutically acceptable salt thereof, administered to the subject is about 500 mg/day. In other embodiments, the amount of the compound having structure (I), or pharmaceutically acceptable salt thereof, administered to the subject is about 600 mg/day. In other embodiments, the amount of the compound having structure (I), or pharmaceutically acceptable salt thereof, administered to the subject is about 700 mg/day. In other embodiments, the amount of the compound having structure (I), or pharmaceutically acceptable salt thereof, administered to the subject is about 800 mg/day. In other embodiments, the amount of the compound having structure (I), or pharmaceutically acceptable salt thereof, administered to the subject is about 900 mg/day. In other embodiments, the amount of the compound having structure (I), or pharmaceutically acceptable salt thereof, administered to the subject is about 1000 mg/day.

In any of the aforementioned embodiments, the method may further comprise administering to said subject one or more additional therapeutic agents.

In any of the aforementioned embodiments, the subject may be an adult or may be a child of less than 18 years of age. In some embodiments, the subject is younger than 18 years of age. In some embodiments, the subject is from 5 to 10 years of age. In some embodiments, the subject is from 6 to 12 years of age. In some embodiments, the subject is from 2 to 6 years of age. In some embodiments of the aforementioned methods, the subject is 8 years old or older.

In some embodiments, the subject is a pediatric subject who is from 2 years of age up to 4 years of age; from 5 years of age up to 7 years of age; or from 8 to 17 years of age.

In some embodiments of the aforementioned uses and methods of treatment, the amount of the compound having structure (I), or pharmaceutically acceptable salt thereof, administered to the subject is from 1 mg/kg to 15 mg/kg per day. In some embodiments, the amount of the compound having structure (I), or pharmaceutically acceptable salt thereof, administered to the subject is from 3 mg/kg to 12 mg/kg per day. In some embodiments, the amount of the compound having structure (I), or pharmaceutically acceptable salt thereof, administered to the subject is from 3 mg/kg to 6 mg/kg per day. In some of these embodiments, the subject is a child (e.g., less than 18 years of age; from 2 to 6 years of age; from 5 to 10 years of age; from 6 to 12 years of age).

In any of the aforementioned embodiments, the amount of the SGLT2 inhibitor administered to the subject may be from about 1 mg/day to about 1000 mg/day. For example, in some embodiments, the amount of the SGLT2 inhibitor administered to the subject is from about 5 mg/day to about 500 mg/day. In some embodiments, the amount of the SGLT2 inhibitor administered to the subject is from about 5 mg/day to about 50 mg/day. In some embodiments, the amount of the SGLT2 inhibitor administered to the subject is 5 mg/day to 50 mg/day. In some embodiments, the amount of the SGLT2 inhibitor administered to the subject is 5 mg/day, 10 mg/day, 12.5 mg/day, or 25 mg/day.

In any of the aforementioned embodiments, the compound having structure (I), or pharmaceutically acceptable salt thereof, and the SGLT2 inhibitor (or pharmaceutical compositions comprising one or both of these ingredients) may be administered on the same day or on different days. In some embodiments, the compound having structure (I), or pharmaceutically acceptable salt thereof, and the SGLT2 inhibitor are administered on the same day. In some embodiments, the compound of structure (I), or pharmaceutically acceptable salt thereof, and the SGLT2 inhibitor are formulated together in the same pharmaceutical composition for simultaneous administration. In some embodiments, the subject is administered the compound having structure (I), or pharmaceutically acceptable salt thereof, and begins treatment with the SGLT2 inhibitor days or weeks after.

In any of the aforementioned embodiments, the compound having structure (I), or pharmaceutically acceptable salt thereof, and the SGLT2 inhibitor, may be administered together in a fixed dose combination formulation.

In any of the aforementioned embodiments, the dose of one or both of the compound having structure (I), or pharmaceutically acceptable salt thereof, and the SGLT2 inhibitor may be lower than when administered without the other. In some embodiments, the dose of the compound having structure (I), or pharmaceutically acceptable salt thereof, is less than 800 mg/day, less than 400 mg/day, or less than 200 mg/day. In some embodiments, the dose of the SGLT2 inhibitor is less than 5 mg/day, 10 mg/day, 12.5 mg/day, or 25 mg/day.

In any of the aforementioned embodiments, the kidney disease or disorder may be a proteinuric disease, such as focal segmental glomerulosclerosis (FSGS), minimal change disease (MCD), immunoglobulin A nephropathy (IgAN), immunoglobulin A-associated vasculitis (IgAV), or Alport syndrome.

In some embodiments, the present disclosure provides a combination therapy as described herein for use in any of the aforementioned methods.

In some embodiments, the present disclosure provides a combination therapy as described herein for use in the manufacture of a medicament for use in any of the aforementioned therapeutic methods.

### Methods of Treating FSGS

In some embodiments, a combination therapy as described herein (such as in any of the aforementioned embodiments) is used to treat a subject having FSGS.

In some embodiments, a method of treating FSGS in a subject in need thereof is provided, the method comprising administering to the subject, over an administration period, the combination therapy in an amount sufficient to achieve or maintain a UP/C ratio of less than or equal to 1.5 g/g for at least a portion of the administration period.

In some embodiments, the FSGS may be primary FSGS. For subjects having primary FSGS, the FSGS is not secondary to other causes such as reduction in renal mass, including that which may be associated with low birth weight; vesicoureteral reflux; obesity; medications; infections, including HIV infection; and systemic illnesses, such as diabetes, sickle cell anemia, and lupus.

In some embodiments, the subject may have FSGS lesions or a genetic mutation in a podocyte protein associated with FSGS.

In some embodiments, the subject is not a pregnant or breastfeeding female. In some embodiments, if the subject is of childbearing potential, the subject is also administered monthly pregnancy tests, and if a pregnancy test indicates that the subject is pregnant, administration of said compound of structure (I) or pharmaceutically acceptable salt thereof, is discontinued. In some embodiments, if the subject is of childbearing potential, the subject is also administered an oral contraceptive, an implanted contraceptive, an injected contraceptive, or an intrauterine device.

In some embodiments, the subject does not have severe hepatic impairment or severe renal impairment. In some embodiments, the subject does not have G5 renal impairment (i.e., GFR<15 mL/min/1.73 m²). In some embodiments, the subject does not have severe hepatic impairment (i.e., Child-Pugh class C).

In some embodiments, the subject is not coadministered one or more of: aliskiren, an angiotensin converting enzyme (ACE) inhibitor, a mineralocorticoid receptor antagonist, a strong CYP3A4/5 inhibitor, a strong CYP3A4/5 inducer, a renin angiotensin aldosterone system (RAAS) inhibitor, and an endothelin system inhibitor.

In some embodiments, the subject may have a baseline eGFR greater than or equal to 30 mL/min/1.73 m² and a baseline urinary protein to creatinine ratio (UP/C) greater than 1.5 g/g. In some embodiments, the subject has a baseline eGFR greater than or equal to 30 mL/min/1.73 m². In some embodiments, the subject has a baseline eGFR of less than 60 mL/min/1.73 m². In some embodiments, the subject has a baseline eGFR of greater than 60 mL/min/1.73 m². In some embodiments, the subject has a baseline UP/C of less than or equal to 2 g/g. In some embodiments, the subject has a baseline UP/C of greater than 3.5 g/g.

### Methods of Treating IgAN

In some embodiments, a combination therapy as described herein (such as in any of the aforementioned embodiments) is used to treat a subject having IgAN.

In some embodiments, a method of treating IgAN in a subject in need thereof is provided, the method comprising administering to the subject, over an administration period, the combination therapy in an amount sufficient to achieve or maintain a UP/C ratio of less than or equal to 1.0 g/g for at least a portion of the administration period.

In some embodiments, the subject has biopsy-confirmed or biopsy-proven primary IgAN.

In some embodiments, the subject may have IgAN that is not secondary to another condition.

In some embodiments, the subject may be currently on a stable dose of ACEI and/or ARB therapy, e.g., for at least 12 weeks prior to treatment with sparsentan. In some embodiments, the subject may be receiving an ACEI and/or ARB at the maximum tolerated dose and at least one-half of the maximum labeled dose.

In some embodiments, the subject may have a systolic BP≤150 mmHg and diastolic BP≤100 mmHg prior to treatment with sparsentan.

In some embodiments, the subject is not a pregnant or breastfeeding female. In some embodiments, if the subject is of childbearing potential, the subject is also administered monthly pregnancy tests, and if a pregnancy test indicates that the subject is pregnant, administration of said compound of structure (I) or pharmaceutically acceptable salt thereof, is discontinued. In some embodiments, if the subject is of childbearing potential, the subject is also administered an oral contraceptive, an implanted contraceptive, an injected contraceptive, or an intrauterine device.

In some embodiments, the subject does not exhibit cellular glomerular crescents in >25% of glomeruli on renal biopsy, e.g., within 6 months of beginning treatment with sparsentan.

In some embodiments, the subject does not have chronic kidney disease (CKD) in addition to IgAN.

In some embodiments, the subject does not have a history of organ transplantation, with the exception of corneal transplants.

In some embodiments, the subject is not treated with systemic immunosuppressive medications (including corticosteroids) for >2 weeks within 3 months of beginning treatment with sparsentan.

In some embodiments, the subject does not have a history of heart failure or previous hospitalization for heart failure or unexplained dyspnea, orthopnea, paroxysmal nocturnal dyspnea, ascites, and/or peripheral edema.

In some embodiments, the subject does not have clinically significant cerebrovascular disease or coronary artery disease within 6 months of beginning treatment with sparsentan.

In some embodiments, the subject does not have a significant hepatic condition or severe hepatic impairment. In some embodiments, the subject does not have severe hepatic impairment (i.e., Child-Pugh class C). In some embodiments, the subject does not have jaundice, hepatitis, or known hepatobiliary disease or elevations of transaminases (ALT/AST)>2 times upper limit of normal prior to beginning treatment with sparsentan.

In some embodiments, the subject does not have a history of malignancy other than adequately treated basal cell or squamous cell skin cancer or cervical carcinoma within the past 2 years prior to beginning treatment with sparsentan.

In some embodiments, the subject does not have a hematocrit value<27% (0.27 V/V), a hemoglobin value<9 g/dL (90 g/L), and/or potassium>5.5 mEq/L (5.5 mmol/L) prior to beginning treatment with sparsentan.

In some embodiments, the subject may have a baseline eGFR greater than or equal to 30 mL/min/1.73 m² and a baseline urinary protein to creatinine ratio (UP/C) greater than or equal to 1.0 g/g.

### Pharmaceutical Formulations

In one aspect, the present disclosure relates to the administration of (i) a pharmaceutical composition comprising the compound of structure (I), or pharmaceutically acceptable salt thereof, and pharmaceutically acceptable excipient, and (ii) a pharmaceutical composition comprising an SGLT2 inhibitor. In some embodiments, the compound of structure (I), or pharmaceutically acceptable salt thereof, and the SGLT2 inhibitor are formulated together in the same pharmaceutical composition, such as in a fixed dose combination unit dose form. Techniques for formulation and administration of the compound of structure (I), or pharmaceutically acceptable salt thereof, and/or the SGLT2 inhibitor, may be found, for example, in “Remington's Pharmaceutical Sciences,” Mack Publishing Co., Easton, PA, 18th edition, 1990, which is incorporated herein by reference for teachings relevant to such techniques. In some embodiments, the pharmaceutical composition is formulated as described below.

In some embodiments, an excipient includes any substance, not itself a therapeutic agent, used as a carrier, diluent, adjuvant, or vehicle for delivery of a therapeutic agent to a subject or added to a pharmaceutical composition to improve its handling or storage properties or to permit or facilitate formation of a dose unit of the composition into a discrete article such as a capsule, tablet, film coated tablet, caplet, gel cap, pill, pellet, bead, and the like suitable for oral administration. For example, an excipient may be a surface active agent (or “surfactant”), carrier, diluent, disintegrant, binding agent, wetting agent, polymer, lubricant, glidant, coating or coating assistant, film forming substance, sweetener, solubilizing agent, smoothing agent, suspension agent, substance added to mask or counteract a disagreeable taste or odor, flavor, colorant, fragrance, or substance added to improve appearance of the composition, or a combination thereof.

Acceptable excipients include, for example, microcrystalline cellulose, lactose, sucrose, starch powder, maize starch or derivatives thereof, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinyl-pyrrolidone, polyvinyl alcohol, saline, dextrose, mannitol, lactose monohydrate, lecithin, albumin, sodium glutamate, cysteine hydrochloride, croscarmellose sodium, sodium starch glycolate, hydroxypropyl cellulose, poloxamer (e.g., poloxamers 101, 105, 108, 122, 123, 124, 181, 182, 183, 184, 185, 188, 212, 215, 217, 231, 234, 235, 237, 238, 282, 284, 288, 331, 333, 334, 335, 338, 401, 402, 403, and 407, and poloxamer 105 benzoate, poloxamer 182 dibenzoate 407, and the like), sodium lauryl sulfate, colloidal silicon dioxide, and the like. Examples of suitable excipients for tablets and capsules include microcrystalline cellulose, silicified microcrystalline cellulose, lactose monohydrate, croscarmellose sodium, sodium starch, hydroxypropyl cellulose, poloxamer 188, sodium lauryl sulfate, colloidal silicon dioxide, and magnesium stearate. Examples of suitable excipients for soft gelatin capsules include vegetable oils, waxes, fats, and semisolid and liquid polyols. Suitable excipients for the preparation of solutions and syrups include, for example, water, polyols, sucrose, invert sugar, and glucose. The compound can also be made in microencapsulated form. If desired, absorption enhancing preparations (for example, liposomes), can be utilized. Acceptable excipients for therapeutic use are well known in the pharmaceutical art, and are described, for example, in “Handbook of Pharmaceutical Excipients,” 5th edition (Raymond C Rowe, Paul J Sheskey and Siân C Owen, eds. 2005), and “Remington: The Science and Practice of Pharmacy,” 21st edition (Lippincott Williams & Wilkins, 2005), which are incorporated herein by reference for teachings relevant to such excipients.

In some embodiments, surfactants are used. Use of surfactants as wetting agents in oral drug forms or to improve the permeation and bioavailability of pharmaceutical active compounds is described in the literature, for example in H. Sucker, P. Fuchs, P. Speiser, Pharmazeutische Technologie, 2nd edition, Thieme 1989, page 260, and Advanced Drug Delivery Reviews (1997), 23, pages 163-183, which are incorporated herein by reference for such teachings. Examples of surfactants include anionic surfactants, non-ionic surfactants, zwitterionic surfactants, and a mixture thereof. In some embodiments, the surfactant is selected from the group consisting of poly(oxyethylene) sorbitan fatty acid ester, poly(oxyethylene) stearate, poly(oxyethylene) alkyl ether, polyglycolated glyceride, poly(oxyethylene) castor oil, sorbitan fatty acid ester, poloxamer, fatty acid salt, bile salt, alkyl sulfate, lecithin, mixed micelle of bile salt and lecithin, glucose ester vitamin E TPGS (D-α-tocopheryl polyethylene glycol 1000 succinate), sodium lauryl sulfate, and the like, and a mixture thereof.

As used herein, the term “carrier” defines a chemical compound that facilitates the incorporation of a compound into cells or tissues. For example, dimethyl sulfoxide (DMSO) is a commonly utilized carrier, as it facilitates the uptake of many organic compounds into the cells or tissues of an organism. As used herein, the term “diluent” defines chemical compounds diluted in water that will dissolve the compound of interest as well as stabilize the biologically active form of the compound. Salts dissolved in buffered solutions are commonly utilized as diluents in the art. One commonly used buffered solution is phosphate buffered saline because it mimics the salt conditions of human blood. Because buffer salts can control the pH of a solution at low concentrations, a buffered diluent rarely modifies the biological activity of a compound. In some embodiments, a diluent selected from one or more of the compounds sucrose, fructose, glucose, galactose, lactose, maltose, invert sugar, calcium carbonate, lactose, starch, microcrystalline cellulose, lactose monohydrate, calcium hydrogen phosphate, anhydrous calcium hydrogen phosphate, a pharmaceutically acceptable polyol such as xylitol, sorbitol, maltitol, mannitol, isomalt, and glycerol, polydextrose, starch, and the like, or any mixture thereof, is used. Acceptable carriers or diluents for therapeutic use are well known in the pharmaceutical art, and are described, for example, in “Remington's Pharmaceutical Sciences,” 18th Ed., Mack Publishing Co., Easton, PA (1990), which is incorporated herein by reference for teachings relevant to such carriers or diluents.

In some embodiments, disintegrants such as starches, clays, celluloses, algins, gums, or crosslinked polymers are used, for example, to facilitate tablet disintegration after administration. Suitable disintegrants include, for example, crosslinked polyvinylpyrrolidone (PVP-XL), sodium starch glycolate, alginic acid, methacrylic acid DYB, microcrystalline cellulose, crospovidone, polacriline potassium, sodium starch glycolate, starch, pregelatinized starch, croscarmellose sodium, and the like. In some embodiments, the formulation can also contain minor amounts of nontoxic auxiliary substances such as wetting or emulsifying agents, pH buffering agents, and the like; for example, sodium acetate, sorbitan monolaurate, triethanolamine sodium acetate, triethanolamine oleate, sodium lauryl sulfate, dioctyl sodium sulfosuccinate, polyoxyethylene sorbitan fatty acid esters, and the like.

In some embodiments, binders are used, for example, to impart cohesive qualities to a formulation, and thus ensure that the resulting dosage form remains intact after compaction. Suitable binder materials include, but are not limited to, microcrystalline cellulose, gelatin, sugars (including, for example, sucrose, glucose, dextrose and maltodextrin), polyethylene glycol, waxes, natural and synthetic gums, polyvinylpyrrolidone, pregelatinized starch, povidone, cellulosic polymers (including, for example, hydroxypropyl cellulose (HPC), hydroxypropyl methylcellulose (HPMC), methyl cellulose, hydroxyethyl cellulose, and the like), and the like. Accordingly, in some embodiments, a formulations disclosed herein includes at least one binder to enhance the compressibility of the major excipient(s). For example, the formulation can include at least one of the following binders in the following ranges: from about 2% to about 6% w/w hydroxypropyl cellulose (Klucel); from about 2% to about 5% w/w polyvinylpyrrolidone (PVP); from about 1% to about 5% w/w methylcellulose; from about 2% to about 5% hydroxypropyl methylcellulose; from about 1% to about 5% w/w ethylcellulose; from about 1% to about 5% w/w sodium carboxy methylcellulose; and the like. One of ordinary skill in the art would recognize additional binders and/or amounts that can be used in the formulations described herein. As would be recognized by one of ordinary skill in the art, when incorporated into the formulations disclosed herein, the amounts of the major filler(s) and/or other excipients can be reduced accordingly to accommodate the amount of binder added in order to keep the overall unit weight of the dosage form unchanged. In some embodiments, a binder is sprayed on from solution, e.g., wet granulation, to increase binding activity.

In some embodiments, a lubricant is employed in the manufacture of certain dosage forms. For example, a lubricant may be employed when producing tablets. In some embodiments, a lubricant can be added just before the tableting step, and can be mixed with the other ingredients for a minimum period of time to obtain good dispersal. In some embodiments, one or more lubricants may be used. Examples of suitable lubricants include magnesium stearate, calcium stearate, zinc stearate, stearic acid, talc, glyceryl behenate, polyethylene glycol, polyethylene oxide polymers (for example, available under the registered trademarks of Carbowax® for polyethylene glycol and Polyox® for polyethylene oxide from Dow Chemical Company, Midland, Mich.), sodium lauryl sulfate, magnesium lauryl sulfate, sodium oleate, sodium stearyl fumarate, DL-leucine, colloidal silica, and others as known in the art. Typical lubricants are magnesium stearate, calcium stearate, zinc stearate, and mixtures of magnesium stearate with sodium lauryl sulfate. Lubricants may comprise from about 0.25% to about 50% of the tablet weight, typically from about 1% to about 40%, more typically from about 5% to about 30%, and most typically from 20% to 30%. In some embodiments, magnesium stearate can be added as a lubricant, for example, to improve powder flow, prevent the blend from adhering to tableting equipment and punch surfaces, and provide lubrication to allow tablets to be cleanly ejected from tablet dies. In some embodiments, magnesium stearate may be added to pharmaceutical formulations at concentrations ranging from about 0.1% to about 5.0% w/w, or from about 0.25% to about 4% w/w, or from about 0.5% w/w to about 3% w/w, or from about 0.75% to about 2% w/w, or from about 0.8% to about 1.5% w/w, or from about 0.85% to about 1.25% w/w, or from about 0.9% to about 1.20% w/w, or from about 0.85% to about 1.15% w/w, or from about 0.90% to about 1.1. % w/w, or from about 0.95% to about 1.05% w/w, or from about 0.95% to about 1% w/w. The above ranges are examples of typical ranges. One of ordinary skill in the art would recognize additional lubricants and/or amounts that can be used in the formulations described herein. As would be recognized by one of ordinary skill in the art, when incorporated into the pharmaceutical compositions disclosed herein, the amounts of the major filler(s) and/or other excipients may be reduced accordingly to accommodate the amount of lubricant(s) added in order to keep the overall unit weight of the dosage form unchanged.

In some embodiments, one or more glidants are used. Examples of glidants include colloidal silicon dioxide, magnesium trisilicate, powdered cellulose, starch, talc, and calcium phosphate, and the like, and mixtures thereof.

In some embodiments, the formulations can include a coating, for example, a film coating. Where film coatings are included, coating preparations may include, for example, a film-forming polymer, a plasticizer, or the like. Also, the coatings may include pigments or opacifiers. Examples of film-forming polymers include hydroxypropyl methylcellulose, hydroxypropyl cellulose, methylcellulose, polyvinyl pyrrolidine, and starches. Examples of plasticizers include polyethylene glycol, tributyl citrate, dibutyl sebacate, castor oil, and acetylated monoglyceride. Furthermore, examples of pigments and opacifiers include iron oxides of various colors, lake dyes of many colors, titanium dioxide, and the like.

In some embodiments, one or more color additives are included. The colorants can be used in amounts sufficient to distinguish dosage form strengths. In some embodiments, color additives approved for use in drugs (see 21 C.F.R. pt. 74) are added to the commercial formulations to differentiate tablet strengths. The use of other pharmaceutically acceptable colorants and combinations thereof is also encompassed by the current disclosure.

The pharmaceutical compositions as disclosed herein may include any other agents that provide improved transfer, delivery, tolerance, and the like. These compositions may include, for example, powders, pastes, jellies, waxes, oils, lipids, lipid (cationic or anionic) containing vesicles (such as Lipofectin®), DNA conjugates, anhydrous absorption pastes, oil-in-water and water-in-oil emulsions, emulsions of Carbowax (polyethylene glycols of various molecular weights), semi-solid gels, and semisolid mixtures containing Carbowax.

In various embodiments, alcohols, esters, sulfated aliphatic alcohols, and the like may be used as surface active agents; sucrose, glucose, lactose, starch, crystallized cellulose, mannitol, light anhydrous silicate, magnesium aluminate, magnesium methasilicate aluminate, synthetic aluminum silicate, calcium carbonate, sodium acid carbonate, calcium hydrogen phosphate, calcium carboxymethyl cellulose, and the like may be used as excipients; magnesium stearate, talc, hardened oil, and the like may be used as smoothing agents; coconut oil, olive oil, sesame oil, peanut oil, and soya may be used as suspension agents or lubricants; cellulose acetate phthalate as a derivative of a carbohydrate such as cellulose or sugar, methyl acetatemethacrylate copolymer as a derivative of polyvinyl, or plasticizers such as ester phthalate may be used as suspension agents.

In some embodiments, a pharmaceutical composition as disclosed herein further comprises one or more of preservatives, stabilizers, dyes, sweeteners, fragrances, flavoring agents, and the like. For example, sodium benzoate, ascorbic acid, and esters of p-hydroxybenzoic acid may be included as preservatives. Antioxidants and suspending agents may also be included in the pharmaceutical composition.

In addition to being used as a monotherapy, the compounds and pharmaceutical compositions disclosed herein may also find use in combination therapies. Effective combination therapy may be achieved with a single pharmaceutical composition that includes multiple active ingredients, or with two or more distinct pharmaceutical compositions. Alternatively, each therapy may precede or follow the other by intervals ranging from minutes to months.

In some embodiments, one or more of, or any combination of, the listed excipients can be specifically included or excluded from the pharmaceutical compositions or methods disclosed herein.

Any of the foregoing formulations may be appropriate in treatments and therapies in accordance with the disclosure herein, provided that the one or more active ingredient in the pharmaceutical composition is not inactivated by the formulation and the formulation is physiologically compatible and tolerable with the route of administration (see also Baldrick P., Regul. Toxicol. Pharmacol. 32 (2): 210-218, 2000; Charman W. N., J. Pharm. Sci. 89 (8): 967-78, 2000, and the citations therein; which references are incorporated herein by reference for teachings relevant to formulations, excipients, and carriers well known to pharmaceutical chemists).

In some embodiments, the above excipients can be present in an amount up to about 95% of the total composition weight, or up to about 85% of the total composition weight, or up to about 75% of the total composition weight, or up to about 65% of the total composition weight, or up to about 55% of the total composition weight, or up to about 45% of the total composition weight, or up to about 43% of the total composition weight, or up to about 40% of the total composition weight, or up to about 35% of the total composition weight, or up to about 30% of the total composition weight, or up to about 25% of the total composition weight, or up to about 20% of the total composition weight, or up to about 15% of the total composition weight, or up to about 10% of the total composition weight, or less.

As will be appreciated by those of skill in the art, the amounts of excipients will be determined by drug dosage and dosage form size. In some embodiments disclosed herein, the dosage form size for providing the compound of structure (I), or a pharmaceutically acceptable salt thereof, is about 100 mg to 800 mg. In some embodiments disclosed herein, the dosage form size is about 100 mg. In some embodiments disclosed herein, the dosage form size is about 200 mg. In some embodiments disclosed herein, the dosage form size is about 400 mg. In some embodiments disclosed herein, the dosage form size is about 800 mg. One skilled in the art will realize that a range of weights may be made and are encompassed by this disclosure.

The pharmaceutical compositions of the present disclosure may be manufactured in a manner that is itself known, e.g., by means of conventional mixing, dissolving, granulating, dragee-making, levigating, emulsifying, encapsulating, entrapping, or tableting processes.

The pharmaceutical compositions of the present disclosure may provide low-dose formulations of the compound of structure (I), or pharmaceutically acceptable salt thereof, or the SGLT2 inhibitor, in tablets, film coated tablets, capsules, caplets, pills, gel caps, pellets, beads, or dragee dosage forms. The formulations disclosed herein can provide favorable drug processing qualities, including, for example, rapid tablet press speeds, reduced compression force, reduced ejection forces, blend uniformity, content uniformity, uniform dispersal of color, accelerated disintegration time, rapid dissolution, low friability (preferable for downstream processing such as packaging, shipping, pick-and-pack, etc.) and dosage form physical characteristics (e.g., weight, hardness, thickness, friability) with little variation.

Proper formulation is dependent upon the route of administration chosen. Suitable routes for administering the compound of structure (I), or pharmaceutically acceptable salt thereof, or the SGLT2 inhibitor, or a pharmaceutical composition comprising the same, may include, for example, oral, rectal, transmucosal, topical, or intestinal administration; and parenteral delivery, including intramuscular, subcutaneous, intravenous, intramedullary injections, intrathecal, direct intraventricular, intraperitoneal, intranasal, or intraocular injections. The compound of structure (I), or pharmaceutically acceptable salt thereof, or the SGLT2 inhibitor, may also be administered in sustained or controlled release dosage forms, including depot injections, osmotic pumps, pills, transdermal (including electrotransport) patches, and the like, for prolonged or timed, pulsed administration at a predetermined rate.

Injectables can be prepared in conventional forms, either as liquid solutions or suspensions, solid forms suitable for solution or suspension in liquid prior to injection, or as emulsions. Suitable excipients may include, for example, water, saline, dextrose, mannitol, lactose, lecithin, albumin, sodium glutamate, cysteine hydrochloride, and the like. In addition, if desired, the injectable pharmaceutical compositions may contain minor amounts of nontoxic auxiliary substances, such as wetting agents, pH buffering agents, and the like. Physiologically compatible buffers include Hanks' solution, Ringer's solution, or physiological saline buffer. If desired, absorption enhancing preparations (for example, liposomes), may be utilized.

For transmucosal administration, penetrants appropriate to the barrier to be permeated may be used in the formulation.

Pharmaceutical formulations for parenteral administration, e.g., by bolus injection or continuous infusion, include aqueous solutions of the active compounds in water-soluble form. Additionally, suspensions of the active compounds may be prepared as appropriate oily injection suspensions. Suitable lipophilic solvents or vehicles include fatty oils such as sesame oil, or other organic oils such as soybean, grapefruit, or almond oils, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions. Formulations for injection may be presented in unit dosage form, e.g., in ampoules or in multi-dose containers, with an added preservative. The compositions may take such forms as suspensions, solutions, or emulsions in oily or aqueous vehicles, and may contain formulatory agents such as suspending, stabilizing, or dispersing agents. Alternatively, the active ingredient may be in powder form for constitution with a suitable vehicle, e.g., sterile pyrogen-free water, before use.

For oral administration, the compound of structure (I), or pharmaceutically acceptable salt thereof, or the SGLT2 inhibitor, can be formulated by combining the active compound with pharmaceutically acceptable carriers known in the art. Such carriers enable the compound to be formulated as tablets, film coated tablets, pills, dragees, capsules, liquids, gels, get caps, pellets, beads, syrups, slurries, suspensions, and the like, for oral ingestion by a subject (e.g., a patient) to be treated.

Pharmaceutical preparations for oral use can be obtained by combining the active compound with solid excipient, optionally grinding a resulting mixture, and processing the mixture of granules, after adding suitable auxiliaries, if desired, to obtain tablets or dragee cores. Suitable excipients may be, in particular, fillers such as sugars, including lactose, sucrose, mannitol, or sorbitol; and cellulose preparations such as, for example, maize starch, wheat starch, rice starch, potato starch, gelatin, gum tragacanth, methyl cellulose, hydroxypropylmethyl-cellulose, sodium carboxymethylcellulose, or polyvinylpyrrolidone (PVP). If desired, disintegrating agents may be added, such as the cross-linked polyvinyl pyrrolidone, agar, or alginic acid or a salt thereof such as sodium alginate. Dragee cores having suitable coatings are also within the scope of the disclosure. For this purpose, concentrated sugar solutions may be used, which may optionally contain gum arabic, talc, polyvinyl pyrrolidone, carbopol gel, polyethylene glycol, titanium dioxide, lacquer solutions, and suitable organic solvents or solvent mixtures. Dyestuffs or pigments may be added to the tablets or dragee coatings for identification or to characterize different combinations of active compound doses. For this purpose, concentrated sugar solutions may be used, which may optionally contain gum arabic, talc, polyvinyl pyrrolidone, carbopol gel, polyethylene glycol, titanium dioxide, lacquer solutions, or suitable organic solvents or solvent mixtures. Dyestuffs or pigments may be added to the tablets or dragee coatings for identification or to characterize different combinations of active compound doses. In addition, stabilizers can be added. In some embodiments, formulations for oral administration are in dosages suitable for such administration. In some embodiments, formulations of the compound of structure (I), or pharmaceutically acceptable salt thereof, or the SGLT2 inhibitor, have an acceptable immediate release dissolution profile and a robust, scalable method of manufacture.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, or lubricants such as talc or magnesium stearate, and, optionally, stabilizers. In soft capsules, the active compounds may be dissolved or suspended in suitable liquids, such as fatty oils, liquid paraffin, or liquid polyethylene glycols. In addition, stabilizers may be added.

For buccal administration, the compositions may take the form of tablets or lozenges formulated in a conventional manner.

For administration by inhalation, the compound of structure (I), or pharmaceutically acceptable salt thereof, or the SGLT2 inhibitor, is conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount. Capsules and cartridges of, e.g., gelatin, for use in an inhaler or insufflator, may be formulated containing a powder mix of the compound and a suitable powder base such as lactose or starch.

Further disclosed herein are various pharmaceutical compositions well known in the pharmaceutical art for uses that include intraocular, intranasal, and intraauricular delivery.

Suitable penetrants for these uses are generally known in the art. Pharmaceutical compositions for intraocular delivery include aqueous ophthalmic solutions of the active compounds in water-soluble form, such as eye drops, or in gellan gum (Shedden et al., Clin. Ther. 23 (3): 440-50, 2001) or hydrogels (Mayer et al., Ophthalmologica 210 (2): 101-3, 1996); ophthalmic ointments; ophthalmic suspensions, such as microparticulates, drug-containing small polymeric particles that are suspended in a liquid carrier medium (Joshi, J. Ocul. Pharmacol. 10 (1): 29-45, 1994), lipid-soluble formulations (Alm et al., Prog. Clin. Biol. Res. 312:447-58, 1989), and microspheres (Mordenti, Toxicol. Sci. 52 (1): 101-6, 1999); and ocular inserts (which references are incorporated herein by reference for teachings relevant to such compositions). Such suitable pharmaceutical formulations may be formulated to be sterile, isotonic, and buffered for stability and comfort. Pharmaceutical compositions for intranasal delivery may also include drops and sprays often prepared to simulate in many respects nasal secretions, to ensure maintenance of normal ciliary action. As disclosed in “Remington's Pharmaceutical Sciences,” 18th Ed., Mack Publishing Co., Easton, PA (1990) (incorporated herein by reference for teachings relevant to such formulations), and well known to those skilled in the art, suitable formulations are most often and preferably isotonic, slightly buffered to maintain a pH of 5.5 to 6.5, and most often and preferably include antimicrobial preservatives and appropriate drug stabilizers. Pharmaceutical formulations for intraauricular delivery include suspensions and ointments for topical application in the ear. Common solvents for such aural formulations include glycerin and water.

The compound of structure (I), or pharmaceutically acceptable salt thereof, or the SGLT2 inhibitor, may also be formulated in rectal compositions such as suppositories or retention enemas, e.g., those containing conventional suppository bases such as cocoa butter or other glycerides.

In addition to the formulations described previously, the compound of structure (I), or pharmaceutically acceptable salt thereof, or the SGLT2 inhibitor, may also be formulated as a depot preparation. Such long-acting formulations may be administered by implantation (for example subcutaneously or intramuscularly) or by intramuscular injection. Thus, for example, the compound of structure (I), or pharmaceutically acceptable salt thereof, or the SGLT2 inhibitor, may be formulated with suitable polymeric or hydrophobic materials (for example, as an emulsion in an acceptable oil) or ion exchange resins, or as sparingly soluble derivatives, for example, as a sparingly soluble salt.

For hydrophobic compounds, a suitable pharmaceutical carrier may be a cosolvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. The proportions of a co-solvent system may be varied considerably without destroying its solubility and toxicity characteristics. Furthermore, the identity of the co-solvent components may be varied: for example, other low-toxicity nonpolar surfactants may be used instead of Polysorbate 80™, the fraction size of polyethylene glycol may be varied; other biocompatible polymers may replace polyethylene glycol, e.g., polyvinyl pyrrolidone; and other sugars or polysaccharides may substitute for dextrose.

Alternatively, other delivery systems for hydrophobic pharmaceutical compounds may be employed. Liposomes and emulsions are well-known examples of delivery vehicles or carriers for hydrophobic drugs. In some embodiments, certain organic solvents such as dimethylsulfoxide also may be employed.

Additionally, the compounds may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Agents intended to be administered intracellularly may be administered using techniques well known to those of ordinary skill in the art. For example, such agents may be encapsulated into liposomes. Molecules present in an aqueous solution at the time of liposome formation are incorporated into the aqueous interior. The liposomal contents are both protected from the external micro-environment and, because liposomes fuse with cell membranes, are efficiently delivered into the cell cytoplasm. The liposome may be coated with a tissue-specific antibody. The liposomes will be targeted to and taken up selectively by the desired organ. Alternatively, small hydrophobic organic molecules may be directly administered intracellularly.

In some embodiments, a solid unit dosage form comprising the compound of structure (I), or pharmaceutically acceptable salt thereof, is provided for use in the compositions and methods described herein. In some embodiments, the solid unit dosage form includes the compound having structure (I), or pharmaceutically acceptable salt thereof, in an amount of 100 mg; about 100 mg; 200 mg; about 200 mg; 400 mg; or about 400 mg. In some embodiments, the solid unit dosage form includes the compound having structure (I), or pharmaceutically acceptable salt thereof, in an amount of 400 mg; or about 400 mg.

In some embodiments, a liquid formulation of the compound of structure (I), or pharmaceutically acceptable salt thereof, is provided for use in the compositions and methods described herein. In some embodiments, the liquid formulation comprises sparsentan and a diluent or vehicle, such as water. In some embodiments, the liquid formulation further comprises (a) a preservative, such as potassium sorbate or sodium benzoate; (b) a sweetener, such as sucralose or sodium saccharin; (c) a flavoring agent; (d) a viscosity modifier such as xanthan gum, microcrystalline cellulose/sodium carboxymethylcellulose composite, methyl cellulose, or hydroxyethyl cellulose; or (e) a pH modifier, such as citric acid, tartaric acid, or sodium citrate; or combinations thereof. For example, in some embodiments, a liquid formulation of sparsentan is provided, which comprises sparsentan, water as a diluent or vehicle, sodium benzoate, sucralose, a flavoring agent, xanthan gum, and citric acid. In some embodiments, the liquid formulation is administered orally to a subject who is 18 years old or younger, 12 years old or younger, from 6 to 12 years of age, or from 2 to 6 years of age.

### Methods of Administration

The combination therapies described herein may be administered to the subject (e.g., a human patient) by any suitable means. Examples of methods of administration include (a) administration though oral pathways, which includes administration in capsule, tablet, granule, spray, syrup, and other such forms; (b) administration through non-oral pathways such as rectal, vaginal, intraurethral, intraocular, intranasal, and intraauricular, which includes administration as an aqueous suspension, an oily preparation, or the like as a drip, spray, suppository, salve, ointment, or the like; (c) administration via injection, subcutaneously, intraperitoneally, intravenously, intramuscularly, intradermally, intraorbitally, intracapsularly, intraspinally, intrasternally, or the like, including infusion pump delivery; (d) administration locally such as by injection directly in the renal or cardiac area, e.g., by depot implantation; and (e) administration topically; as deemed appropriate by those of skill in the art for bringing the compound of structure (I), or pharmaceutically acceptable salt thereof, or the SGLT2 inhibitor into contact with living tissue.

Pharmaceutical compositions suitable for administration include compositions where the compound of structure (I), or pharmaceutically acceptable salt thereof, or the SGLT2 inhibitor is contained in an amount effective to achieve its intended purpose. The dose can be tailored to achieve a desired effect, but will depend on such factors as weight, diet, concurrent medication, and other factors that those skilled in the medical arts will recognize.

In some embodiments, the combination therapy will be administered for a period of continuous therapy, for example for a week or more, or for months or years. In some embodiments, the compound of structure (I), or pharmaceutically acceptable salt thereof, will be administered once daily as long as eGFR is >20 ml/min/1.73 m² or the subject develops end-stage renal disease (ESRD) requiring renal replacement therapy (RRT).

In some embodiments, the dosing regimen of the compound of structure (I), or pharmaceutically acceptable salt thereof, is administered for a period of time, which time period can be, for example, from at least about 4 weeks to at least about 8 weeks, from at least about 4 weeks to at least about 12 weeks, from at least about 4 weeks to at least about 16 weeks, or longer. In some embodiments, the dosing regimen of the compound of structure (I), or pharmaceutically acceptable salt thereof, is administered for 36 weeks or longer. In some embodiments, the dosing regimen of the compound of structure (I), or pharmaceutically acceptable salt thereof, is administered for 36 weeks. In some embodiments, the dosing regimen of the compound of structure (I), or pharmaceutically acceptable salt thereof, is administered for 108 weeks. In some embodiments, the dosing regimen of the compound of structure (I), or pharmaceutically acceptable salt thereof, is administered for 110 weeks. The dosing regimen of the compound of structure (I), or pharmaceutically acceptable salt thereof can be administered three times a day, twice a day, daily, every other day, three times a week, every other week, three times per month, once monthly, substantially continuously, or continuously.

In some embodiments, the dosing regimen of the SGLT2 inhibitor is administered for a period of time, which time period can be, for example, from at least about 4 weeks to at least about 8 weeks, from at least about 4 weeks to at least about 12 weeks, from at least about 4 weeks to at least about 16 weeks, for 36 weeks, for 108 weeks, for 110 weeks, or longer. The dosing regimen of the SGLT2 inhibitor can be administered three times a day, twice a day, daily, every other day, three times a week, every other week, three times per month, once monthly, substantially continuously, or continuously.

In some embodiments, the compound of structure (I), or pharmaceutically acceptable salt thereof, and the SGLT2 inhibitor are administered daily on a continuous or ongoing basis, e.g., for treating a chronic disease or disorder.

In cases of local administration or selective uptake, the effective local concentration of the drug may not be related to plasma concentration. The amount of composition administered may be dependent on the subject being treated, on the subject's weight, the severity of the affliction, and the manner of administration.

In some embodiments, the present disclosure relates combination therapies that include administering to the subject a dosage of the compound of structure (I), or pharmaceutically acceptable salt thereof containing an amount of about 10 mg to about 1000 mg, of drug per dose, orally, at a frequency of three times per month, once monthly, once weekly, once every three days, once every two days, once per day, twice per day, three times per day, substantially continuously, or continuously, for the desired duration of treatment.

In some embodiments, the present disclosure provides a method of using the compound of structure (I), or pharmaceutically acceptable salt thereof, in the treatment of FSGS or IgAN in a subject comprising administering to the subject a dosage containing an amount of about 100 mg to about 1000 mg, of drug per dose, orally, at a frequency of three times per month, once monthly, once weekly, once every three days, once every two days, once per day, twice per day, or three times per day, for the desired duration of treatment.

In some further embodiments, the present disclosure provides a method of using the compound of structure (I), or pharmaceutically acceptable salt thereof, in the treatment of FSGS or IgAN in a subject comprising administering to the subject a dosage containing an amount of about 200 mg of drug per dose, orally, at a frequency of three times per month, once monthly, once weekly, once every three days, once every two days, once per day, twice per day, or three times per day, for the desired duration of treatment.

In some embodiments, the present disclosure provides a method of using the compound of structure (I), or pharmaceutically acceptable salt thereof, in the treatment of FSGS or IgAN in a subject comprising administering to the subject a dosage containing an amount of about 400 mg of drug per dose, orally, at a frequency of three times per month, once monthly, once weekly, once every three days, once every two days, once per day, twice per day, or three times per day, for the desired duration of treatment.

In some embodiments, the present disclosure provides a method of using the compound of structure (I), or pharmaceutically acceptable salt thereof, in the treatment of FSGS or IgAN in a subject comprising administering to the subject a dosage containing an amount of about 800 mg of drug per dose, orally, at a frequency of three times per month, once monthly, once weekly, once every three days, once every two days, once per day, twice per day, or three times per day, for the desired duration of treatment.

In some embodiments, the present disclosure provides a method of using the compound of structure (I), or pharmaceutically acceptable salt thereof, in the treatment of FSGS or IgAN in a subject comprising administering to the subject a dosage from about 0.1 mg/kg to about 100 mg/kg, or from about 0.2 mg/kg to about 50 mg/kg, or from about 0.5 mg/kg to about 25 mg/kg of body weight (or from about 1 mg to about 2500 mg, or from about 100 mg to about 800 mg) of active compound per day, which may be administered in a single dose or in the form of individual divided doses, such as from 1 to 4 times per day. In some embodiments, the amount of the compound of structure (I), or pharmaceutically acceptable salt thereof, administered to the subject is from about 1 mg/kg to about 15 mg/kg, from about 3 mg/kg to about 12 mg/kg, or from about 3 mg/kg to about 6 mg/kg, per day, which may be administered in a single dose or in the form of individual divided doses, such as from 1 to 4 times per day.

In some embodiments of the aforementioned pharmaceutical compositions and methods, the pharmaceutical composition is a solid unit dosage form. In some embodiments, the solid unit dosage form includes the compound having structure (I), or pharmaceutically acceptable salt thereof, in an amount of 100 mg, 200 mg, or 400 mg, or about 100 mg, about 200 mg, or about 400 mg. In some embodiments, the solid unit dosage form includes the compound having structure (I), or pharmaceutically acceptable salt thereof, in an amount of 400 mg, or about 400 mg. In some embodiments, the solid unit dosage form is administered once daily. In some embodiments, the solid unit dosage form is administered orally.

In some embodiments of the aforementioned pharmaceutical compositions and methods, the pharmaceutical composition is a liquid formulation for oral administration. In some particular embodiments, the liquid formulation is administered to a subject who is less than 18 years of age (e.g., from 2 to 6 years of age).

In some embodiments of the aforementioned pharmaceutical compositions and methods, the pharmaceutical composition is formulated for oral administration and is administered with or without food.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in a form prescribed by a governmental agency regulating the manufacture, use, or sale of pharmaceuticals, which notice is reflective of approval by the agency of the form of the drug for human or veterinary administration. Such notice, for example, may be the labeling approved by the United States Food and Drug Administration for prescription drugs, or the approved product insert. Compositions comprising the compound of structure (I), or pharmaceutically acceptable salt thereof, formulated in a compatible pharmaceutical carrier may also be prepared, placed in an appropriate container, and labeled for treatment of an indicated condition.

### EXEMPLARY EMBODIMENTS

The present disclosure also provides in further embodiments:
1. A method of treating a kidney disease or disorder in a subject in need thereof, the method comprising administering to said subject (i) sparsentan, and (ii) a SGLT2 inhibitor.
2. A method of treating a proteinuric disease in a subject in need thereof, the method comprising administering to said subject (i) sparsentan, and (ii) a SGLT2 inhibitor.
3. A method of reducing proteinuria in a subject in need thereof, the method comprising administering to said subject (i) sparsentan, and (ii) a SGLT2 inhibitor. 4. The method according to embodiment 3, wherein the subject has a kidney disease or disorder.
5. The method according to embodiment 3 or embodiment 4, wherein proteinuria is reduced following administration of sparsentan and the SGLT2 inhibitor, and the reduction is greater relative to that of a subject who is not administered the SGLT2 inhibitor.
6. The method according to any one of embodiments 3-5, wherein proteinuria is measured by urinary protein to creatinine ratio (UP/C).
7. The method according to embodiment 6, wherein UP/C is reduced following administration of sparsentan and the SGLT2 inhibitor, and the reduction is greater relative to that of a subject who is not administered the SGLT2 inhibitor.
8. A method of prolonging the time to end stage renal disease in a subject having a kidney disease or disorder, the method comprising administering to said subject (i) sparsentan, and (ii) a SGLT2 inhibitor.
9. The method according to embodiment 8, wherein the time to end stage renal disease is increased following administration of sparsentan and the SGLT2 inhibitor, and the increase is greater relative to that of a subject who is not administered the SGLT2 inhibitor.
10. A method for reducing a risk of end stage renal disease in a subject having a kidney disease or disorder, the method comprising administering to said subject (i) sparsentan, and (ii) a SGLT2 inhibitor.
11. The method according to embodiment 10, wherein the risk of end stage renal disease is reduced following administration of sparsentan and the SGLT2 inhibitor, and the reduction is greater relative to that of a subject who is not administered the SGLT2 inhibitor.
12. A method of inducing complete remission in a subject having a kidney disease or disorder, the method comprising administering to said subject (i) sparsentan, and (ii) a SGLT2 inhibitor.
13. The method according to embodiment 12, wherein urinary protein to creatinine ratio (UP/C) is reduced to less than or equal to 0.3 g/g following administration.
14. A method of reducing the rate of decline in estimated glomerular filtration rate (eGFR) in a subject having a kidney disease or disorder, the method comprising administering to said subject (i) sparsentan, and (ii) a SGLT2 inhibitor.
15. The method according to embodiment 14, wherein the rate of decline in eGFR is reduced following administration of sparsentan and the SGLT2 inhibitor, and the reduction is greater relative to that of a subject who is not administered the SGLT2 inhibitor.
16. The method according to any one of embodiments 1-15, wherein said SGLT2 inhibitor is dapagliflozin, empagliflozin, canagliflozin, ertugliflozin, ipragliflozin, tofogliflozin, or luseogliflozin, or a pharmaceutically acceptable salt, solvate, complex, or salt of solvates thereof.
17. The method according to any one of embodiments 1-15, wherein said SGLT2 inhibitor is dapagliflozin, or a pharmaceutically acceptable salt, solvate, complex, or salt of solvates thereof.
18. The method according to any one of embodiments 1-15, wherein said SGLT2 inhibitor is empagliflozin, or a pharmaceutically acceptable salt, solvate, complex, or salt of solvates thereof.
19. The method according to any one of embodiments 1-18, wherein the amount of sparsentan administered to said subject is from 50 mg/day to 1000 mg/day.
20. The method according to any one of embodiments 1-18, wherein the amount of sparsentan administered to said subject is 100 mg/day.
21. The method according to any one of embodiments 1-18, wherein the amount of sparsentan administered to said subject is 200 mg/day.
22. The method according to any one of embodiments 1-18, wherein the amount of sparsentan administered to said subject is 400 mg/day.
23. The method according to any one of embodiments 1-18, wherein the amount of sparsentan administered to said subject is 800 mg/day.
24. The method according to any one of embodiments 1-18, wherein the amount of sparsentan administered to said subject is 200 mg/day for the first 2 weeks and thereafter 400 mg/day.
25. The method according to any one of embodiments 1-18, wherein if said subject's weight is from 20 kg to 50 kg, the amount of sparsentan administered to said subject is 100 mg/day for the first 2 weeks and thereafter 200 mg/day.
26. The method according to any one of embodiments 1-18, wherein the amount of sparsentan administered to said subject is 400 mg/day for the first 2 weeks and thereafter 800 mg/day.
27. The method according to any one of embodiments 1-18, wherein (a) if said subject's weight is from 20 kg to 50 kg, the amount of sparsentan administered to said subject is 200 mg/day for the first 2 weeks and thereafter 400 mg/day, and (b) if said subject's weight is greater than 50 kg, the amount of sparsentan administered to said subject is 400 mg/day for the first 2 weeks and thereafter 800 mg/day.
28. The method according to any one of embodiments 1-27, wherein said sparsentan is administered orally.
29. The method according to embodiment 28, wherein said sparsentan is administered with or without food.
30. The method according to any one of embodiments 1-29, wherein the amount of said SGLT2 inhibitor administered to said subject is from 1 mg/day to 1000 mg/day.
31. The method according to any one of embodiments 1-29, wherein the amount of said SGLT2 inhibitor administered to said subject is from 1 mg/day to 500 mg/day.
32. The method according to any one of embodiments 1-29, wherein the amount of said SGLT2 inhibitor administered to said subject is from 5 mg/day to 50 mg/day.
33. The method according to any one of embodiments 1-29, wherein the amount of said SGLT2 inhibitor administered to said subject is 5 mg/day, 10 mg/day, 12.5 mg/day, or 25 mg/day.
34. The method according to any one of embodiments 1-29, wherein the amount of said SGLT2 inhibitor administered to said subject is 5 mg/day.
35. The method according to any one of embodiments 1-29, wherein the amount of said SGLT2 inhibitor administered to said subject is 10 mg/day.
36. The method according to any one of embodiments 1-29, wherein the amount of said SGLT2 inhibitor administered to said subject is 25 mg/day.
37. The method according to any one of embodiments 1-36, wherein said SGLT2 inhibitor is administered orally.
38. The method according to embodiment 37, wherein said SGLT2 inhibitor is administered with or without food.
39. The method according to any one of embodiments 1-38, wherein said sparsentan and said SGLT2 inhibitor are administered to said subject as a fixed dose combination formulation.
40. The method according to any one of embodiments 1-39, wherein said kidney disease or disorder or proteinuric disease is focal segmental glomerulosclerosis (FSGS), minimal change disease (MCD), immunoglobulin A nephropathy (IgAN), immunoglobulin A-associated vasculitis (IgAV), or Alport syndrome.
41. The method according to any one of embodiments 1-40, wherein said subject is not a pregnant or breastfeeding female.
42. The method according to any one of embodiments 1-41, wherein if said subject is a female of childbearing potential, said method further comprises administering to such subject monthly pregnancy tests and if a pregnancy test indicates that said subject is pregnant, discontinuing administration of sparsentan.
43. The method according to any one of embodiments 1-42, wherein if said subject is a female of childbearing potential, said method further comprises administering to such subject an oral contraceptive, an implanted contraceptive, an injected contraceptive, or an intrauterine device.
44. The method according to any one of embodiments 1-43, wherein said subject does not have severe hepatic impairment.
45. The method according to any one of embodiments 1-44, wherein said subject does not have severe renal impairment.
46. The method according to any one of embodiments 1-45, wherein said kidney disease or disorder is immunoglobulin A nephropathy (IgAN).
47. The method according to embodiment 46, wherein said subject (i) has a baseline eGFR greater than or equal to 30 mL/min/1.73 m² and (ii) has a baseline UP/C greater than 1.0 g/g.
48. The method according to embodiment 46, wherein said subject (i) has a baseline eGFR greater than or equal to 25 mL/min/1.73 m² and (ii) has a baseline urine albumin-to-creatinine ratio (UA/C) greater than 0.3 g/g.
49. The method according to any one of embodiments 46-48, wherein said subject has biopsy-proven primary IgAN.
50. The method according to any one of embodiments 46-49, wherein said subject has a UP/C of less than or equal to 1.0 g/g following 36 weeks of treatment.
51. The method according to any one of embodiments 46-50, wherein said subject has a UA/C of less than 0.2 g/g following 24 weeks of treatment.
52. The method according to any one of embodiments 46-51, wherein said subject's UA/C is reduced by 30% to 50% from baseline UA/C following 24 weeks of treatment.
53. The method according to any one of embodiments 1-45, wherein said kidney disease or disorder is focal segmental glomerulosclerosis (FSGS).
54. The method according to embodiment 53, wherein said subject (i) has a baseline estimated glomerular filtration rate (eGFR) greater than or equal to 30 mL/min/1.73 m² and (ii) has a baseline UP/C greater than 1.5 g/g.
55. The method according to embodiment 53 or embodiment 54, wherein the FSGS is primary FSGS.
56. The method according to any one of embodiments 53-55, wherein said subject is 8 years of age or older.
57. The method according to any one of embodiments 53-56, wherein said subject has FSGS lesions or a genetic mutation in a podocyte protein associated with FSGS.
58. The method according to any one of embodiments 53-57, wherein said subject has a baseline eGFR of less than 60 mL/min/1.73 m².
59. The method according to any one of embodiments 53-57, wherein said subject has a baseline eGFR of greater than 60 mL/min/1.73 m².
60. The method according to any one of embodiments 53-59, wherein said subject has a baseline UP/C of less than or equal to 2 g/g.
61. The method according to any one of embodiments 53-59, wherein said subject has a baseline UP/C of less than or equal to 3.5 g/g.
62. The method according to any one of embodiments 53-59, wherein said subject has a baseline UP/C of greater than 3.5 g/g.
63. The method according to any one of embodiments 53-62, wherein said subject is not coadministered aliskiren.
64. The method according to any one of embodiments 53-62, wherein said subject is not coadministered an ACE inhibitor and a mineralocorticoid receptor antagonist.
65. The method according to any one of embodiments 53-64, wherein said subject is not coadministered a strong CYP3A4/5 inhibitor.
66. The method according to any one of embodiments 53-65, wherein said subject is not coadministered a strong CYP3A4/5 inducer.
67. The method according to any one of embodiments 53-66, wherein said subject is not coadministered a renin angiotensin aldosterone system (RAAS) inhibitor or endothelin system inhibitor.
68. The method according to any one of embodiments 53-67, wherein said subject has a UP/C of less than or equal to 1.5 g/g following 36 weeks of treatment.
69. The method according to any one of embodiments 53-68, wherein said subject's UP/C is reduced by more than 40% from the baseline UP/C following 36 weeks of treatment.
70. The method according to any one of embodiments 1-45, wherein said kidney disease or disorder is Alport syndrome.
71. The method according to any one of embodiments 1-70, wherein said subject is administered one or more additional therapeutic agents.
72. Sparsentan for use in the method according to any one of embodiments 1-71, wherein the subject is also administered a SGLT2 inhibitor according to any one of embodiments 1-71.
73. Use of sparsentan and a SGLT2 inhibitor in the manufacture of a medicament for use in the method according to any one of embodiments 1-71.
74. Use of sparsentan in the manufacture of a first medicament, and use of a SGLT2 inhibitor in the manufacture of a second medicament, wherein the first medicament and the second medicament are for use in the method according to any one of embodiments 1-71.
75. A kit for use in the method according to any one of embodiments 1-71, comprising: a pharmaceutical composition comprising sparsentan; and a pharmaceutical composition comprising a SGLT2 inhibitor.

### EXAMPLES

### Example 1

### Treatment of Immunoglobulin a Nephropathy (IgAN) with Sparsentan

The long-term nephroprotective potential of treatment with sparsentan was evaluated in PROTECT, a 114-week, randomized, global, multicenter, double-blind, parallel-group, active-control Phase 3 clinical study in patients with IgAN who have persistent overt proteinuria and remain at high risk of disease progression despite being on a stable dose (or doses) of an angiotensin-converting enzyme inhibitor (ACEI) and/or angiotensin receptor blocker (ARB) that is (are) a maximum tolerated dose that is at least one half of the maximum labeled dose (MLD) (according to approved labeling). The purpose of the study is to evaluate the potential benefit of sparsentan on kidney function by analyzing change in proteinuria (protein in urine) and estimated glomerular filtration rate (eGFR) as compared to current standard treatment.

The study has an open-label extension period of up to 156 weeks, for a total duration of up to 270 weeks.

### Enrollment and Inclusion and Exclusion Criteria

404 patients aged≥18 years were enrolled in the study globally.

Enrolled patients were at high risk of progressing to renal failure. The trial enrolled patients with eGFR≥30 mL/min/1.73 m² and urinary protein to creatinine ratio (UP/C)≥1.0 g/g at screening.

Inclusion criteria included: age 18 years or older at screening; biopsy-proven primary IgAN; proteinuria (UP/C) of ≥1 g/day at screening; eGFR≥30 mL/min/1.73 m² at screening; currently on stable dose of ACEI and/or ARB therapy, for at least 12 weeks prior to screening (maximum tolerated dose and at least one-half of the maximum labeled dose); systolic BP≤150 mmHg and diastolic BP≤100 mmHg at screening; willing to undergo change in ACEI and/or ARB and anti-hypertensive medications; and agree to contraception.

Exclusion criteria for the double-blind period included: IgAN secondary to another condition; presence of cellular glomerular crescents in >25% of glomeruli on renal biopsy (if biopsy available within 6 months of screening); chronic kidney disease (CKD) in addition to IgAN; history of organ transplantation, with exception of corneal transplants; require any prohibited medications; treatment of systemic immunosuppressive medications (including corticosteroids) for >2 weeks within 3 months of screening; history of heart failure or previous hospitalization for heart failure or unexplained dyspnea, orthopnea, paroxysmal nocturnal dyspnea, ascites, and/or peripheral edema; clinically significant cerebrovascular disease or coronary artery disease within 6 months of screening; jaundice, hepatitis, or known hepatobiliary disease or elevations of transaminases (ALT/AST)>2 times upper limit of normal at screening; history of malignancy other than adequately treated basal cell or squamous cell skin cancer or cervical carcinoma within the past 2 years; hematocrit value<27% (0.27 V/V) or hemoglobin value<9 g/dL (90 g/L) at screening; potassium>5.5 mEq/L (5.5 mmol/L) at screening; history of alcohol of illicit drug use disorder; history of serious side effect or allergic response to any angiotensin II antagonist or endothelin receptor antagonist, including sparsentan or irbesartan, or has a hypersensitivity to any of the excipients in the study medications; female patient is pregnant, planning to become pregnant during the course of the study, or breastfeeding; and participation in a study of another investigational product within 28 days of screening.

Exclusion criteria for the open-label extension period based on assessments at the Week 110 and Week 114 visits included: progression to end-stage renal disease (ESRD) requiring renal replacement therapy (RRT); development of any criteria for discontinuation of study medication or discontinuation from the study, between Week 110 and Week 114; patient was unable to initiate, or developed contraindications to, treatment with RAAS inhibitors between Week 110 and Week 114; eGFR≤20 mL/min/1.73 m² at Week 110; and female patient is pregnant or breastfeeding.

Randomized Trial with Administration of Sparsentan

All patients were randomized at a 1:1 ratio to receive sparsentan or the active control, irbesartan (an angiotensin receptor blocker), as a single oral morning dose. Sparsentan was administered at a dose of 200 mg once daily by mouth for 2 weeks and then titrated to 400 mg once daily as tolerated. Irbesartan was administered at a dose of 150 mg once daily by mouth for 2 weeks and then titrated to 300 mg once daily as tolerated.

Sparsentan was provided as a tablet, comprising 200 mg or 400 mg of sparsentan. Inactive ingredients included silicified microcrystalline cellulose, lactose anhydrous, sodium starch glycolate, colloidal silicon dioxide, and magnesium stearate.

Change in proteinuria (UP/C) from baseline (Day 1) at Week 36 in sparsentan-treated patients as compared to irbesartan-treated patients was assessed. The Week 36 UP/C was based on a 24-hour urine sample. Secondary outcome measures included eGFR over a 52-week period and a 104-week period following the initial acute effect of randomized therapy. The initial acute effect of randomized therapy is defined as the first 6 weeks of randomized treatment with study medication; thus, the analysis is from 6 weeks postrandomization to 58 weeks (eGFR chronic slope at 1 year) or 110 weeks (eGFR chronic slope at 2 years) postrandomization, respectively. Also assessed was eGFR over a 58-week and a 110-week (approximately 2-year) period following the initiation of randomized therapy (the analysis is from Day 1 to 58 weeks postrandomization or from Day 1 to 110 weeks postrandomization, eGFR total slope at 2 years).

The study provided for an analysis of topline results at 36 weeks, following the first 280 patients reaching 36 weeks of treatment. After 36 weeks of treatment, patients receiving sparsentan achieved a mean reduction in proteinuria from baseline of 49.8%, compared to a mean reduction in proteinuria from baseline of 15.1% for irbesartan-treated patients (p<0.0001), i.e., the sparsentan-treated group demonstrated a greater than threefold reduction in proteinuria compared to the irbesartan-treated group. Preliminary results from the interim analysis suggest that to date in the study, sparsentan has been generally well-tolerated and consistent with the observed safety profile to date. Preliminary eGFR data available at the time of the interim analysis were indicative of a potential clinically meaningful treatment effect after two years of treatment.

Open Label Extension Period with Combination Therapy of Sparsentan and SGLT2 Inhibitor

6 patients were administered 400 mg of sparsentan and 5 mg or 10 mg of dapagliflozin/FARXIGA® per day, after completing the double-blind portion of the trial.

3 patients were administered 400 mg of sparsentan and 10 mg, 12.5 mg, or 25 mg of empagliflozin/JARDIANCE® per day, after completing the double-blind portion of the trial.

### Example 2

### Treatment of Focal Segmental Glomerulosclerosis with Sparsentan

The efficacy and safety of sparsentan were evaluated in DUPLEX, a randomized, double-blind, active-controlled, multicenter, global trial in patients with FSGS after exclusion of known secondary causes.

### Enrollment and Inclusion and Exclusion Criteria

The trial enrolled patients with eGFR≥30 mL/min/1.73 m² and urinary protein to creatinine ratio (UP/C)>1.5 g/g. Randomization was stratified based on eGFR (≥30 to <60 mL/min/1.73 m² versus eGFR≥60 mL/min/1.73 m²) and UP/C values (≤3.5 g/g versus >3.5 g/g for patients≥18 years of age, or ≤2 g/g versus >2 g/g for patients<18 years of age).

Randomized Trial with Administration of Sparsentan

All patients were randomized at a 1:1 ratio to receive sparsentan or the active control, irbesartan. Sparsentan was administered at a dose of 400 mg once daily by mouth for 2 weeks and then titrated to 800 mg once daily as tolerated. Irbesartan was administered at a dose of 150 mg once daily by mouth for 2 weeks and then titrated to 300 mg once daily as tolerated. In patients weighing 50 kg or less, dosing was halved in both treatment arms (e.g., sparsentan administered at a dose of 200 mg once daily by mouth for 2 weeks and then titrated to 400 mg once daily as tolerated).

Sparsentan was provided as a tablet, comprising 200 mg or 400 mg of sparsentan. Inactive ingredients included silicified microcrystalline cellulose, lactose anhydrous, sodium starch glycolate, colloidal silicon dioxide, and magnesium stearate.

The proportion of patients achieving a UP/C≤1.5 g/g and a >40% reduction from baseline in UP/C was evaluated after patients completed 36 weeks of treatment.

The proportion of patients achieving the composite endpoint of UP/C≤1.5 g/g and a >40% reduction from baseline in UP/C was significantly higher in the sparsentan arm (mean 42.0, std. error 4.73) than in the irbesartan arm (mean 26.0, std. error 4.41) at Week 36 (p=0.0094).

In the DUPLEX Study, a total of 371 patients were randomized 1:1 to receive either sparsentan or irbesartan, the active control. The study protocol provided for an unblinded analysis to evaluate the interim efficacy endpoint—the proportion of patients achieving FPRE, which is a clinically meaningful endpoint defined as urine protein-to-creatinine ratio (UP/C)≤1.5 g/g and a >40 percent reduction in UP/C from Baseline, at Week 36—following the first approximately 190 patients reaching 36 weeks of treatment. After 36 weeks of treatment, 42.0 percent of patients receiving sparsentan achieved FPRE, compared to 26.0 percent of irbesartan-treated patients (p=0.0094).

A preliminary review of the interim safety results indicated that sparsentan has been generally well-tolerated and the overall safety profiles in the study to date have been generally comparable between treatment groups.

Open Label Extension Period with Combination Therapy of Sparsentan and SGLT2 Inhibitor

1 patient was administered 800 mg of sparsentan and 5 mg of dapagliflozin/FARXIGA® per day, after completing the double-blind portion of the trial.

1 patient was administered 800 mg of sparsentan and 10 mg of empagliflozin/JARDIANCE® per day, after completing the double-blind portion of the trial.

### Example 3

### Effect of Multiple Doses of Sparsentan on the Single-Dose Pharmacokinetics of Dapagliflozin: Open-Label Drug-Drug Interaction Study in Healthy Adults

Potential drug-drug interactions (DDI) of sparsentan with SGLT2 inhibitors are not known. The safety of administering sparsentan and dapagliflozin was evaluated in an open-label, drug-drug interaction study in healthy results. This phase 1 study examined the effect of multiple doses of sparsentan on the pharmacokinetics (PK) of single-dose dapagliflozin and assessed the safety and tolerability of single-dose dapagliflozin when co-administered after multiple doses of sparsentan in healthy adults.

The open-label DDI study included Period 1 (Days 1-5; single dose of 10 mg dapagliflozin (“DAPA”) on Day 1, PK sampling pre-dose and up to 96 hours post-dose) and Period 2 (Days 5-14; 800 mg sparsentan (“SPAR”) once daily for 10 days with single 10 mg dose DAPA co-administered on Day 11, DAPA PK sampling pre-dose and up to 96 hours after Day 11 dosing). To avoid hypoglycemic events, subjects received an oral 20% glucose solution in water with the DAPA dose and every ~15 minutes up to 4 hours post-dose. Plasma concentrations and PK parameters of DAPA and SPAR were summarized. Subjects with evaluable data for both periods were included in an ANOVA mixed model analysis of DAPA AUC0-t, AUC0-inf, and Cmax following DAPA+SPAR vs DAPA alone. Treatment-emergent adverse events (TEAEs) were summarized.

Twenty-two healthy adults were enrolled and 20 completed both study periods. Mean peak and extent of DAPA exposure (Cmax and AUC) values were similar following 10 mg DAPA alone and 10 mg DAPA+800 mg SPAR (see Table 1). TEAEs were reported by 14 (63.6%) subjects; most frequent were headache (6; 27.3%), nausea (5; 22.7%), and preprandial asymptomatic hypoglycemia (5; 22.7%).

Multiple dosing of SPAR did not affect single-dose DAPA PK in healthy adults. Single-dose DAPA co-administered with multiple doses of SPAR appeared to be well tolerated by healthy adults.

TABLE 1
Plasma Dapagliflozin PK Parameters Following
Dapagliflozin (“DAPA”) + Sparsentan
(“SPAR”) Versus Dapagliflozin Alone

| | DAPA + | | | | |
|---|---|---|---|---|---|
| | SPAR | DAPA | | | Intra- |
| | Geometric | Geometric | GLSMR | | subject |
| | LSMs | LSMs | (%) | 90% CI | CV % |
| AUC0-t | 526.1 | 508.8 | 103.4 | 99.9-107.0 | 6.3 |
| (ng*hr/mL) | (n = 20) | (n = 20) | | | |
| AUC0-inf | 565.4 | 533.7 | 105.9 | 103.1-108.9 | 4.4 |
| (ng*hr/mL) | (n = 16) | (n = 16) | | | |
| Cmax | 79.9 | 73.9 | 108.2 | 96.0-121.8 | 22.0 |
| (ng/mL) | (n = 20) | (n = 20) | | | |

### Example 4

### Treatment of Immunoglobulin a Nephropathy (IgAN) with Sparsentan and a Sglt2 Inhibitor

The efficacy and safety of sparsentan in combination with SGLT2 inhibition in adult participants with IgAN are evaluated in a multicentered, single-group Phase 2, exploratory, open-label clinical study.

### Overall Design

The trial is a 28-week, open-label, multicenter, single-group Phase 2 exploratory study to determine the safety and effect of sparsentan in participants with IgAN who are at risk of disease progression to kidney failure despite being on both stable renin-angiotensin-aldosterone system inhibitor (RAASi) and sodium glucose cotransporter-2 (SGLT2) inhibitor treatment for at least 12 weeks prior to study entry.

This study includes 3 periods: a screening period (up to 42 days), a treatment period (24 weeks) and a follow-up period (4 weeks). All participants are required to be on a stable dose(s) of angiotensin converting enzyme inhibitor (ACEI) and/or angiotensin receptor blocker (ARB) therapy and on a stable dose of a SGLT2 inhibitor at screening and will continue their stable treatments through the screening period. Eligible participants entering the study will remain on the stable dose of the SGLT2 inhibitor for the duration of the study, however, they will discontinue ACEI and/or ARB therapy before the Day 1 visit. The final dose of an ACEI and/or ARB therapy should be taken on the day before the Day 1 visit. Baseline evaluations will be taken on Day 1 prior to administration of the first dose of the study intervention with sparsentan.

The full daily dose of study intervention (sparsentan) is preferred to be taken prior to the morning meal, with the exception of the day of a study visit. Allowed doses of sparsentan during the treatment period are 200 mg and 400 mg. The goal is to titrate from the initial dose of 200 mg (Day 1) to the target dose of 400 mg at Week 2.

Planned maximum duration for each participant in this study is 34 weeks.

Study visits will be conducted at weeks 2-, 4-, 12-, and 24-following Day 1. Following the 24-week treatment period, study intervention will be discontinued for 4 weeks followed by a return to standard of care. Participants will return to the site for a visit at Week 28 for the safety follow-up visit.

Urine albumin-to-creatinine ratio (UA/C) and urine protein-to-creatinine ratio (UP/C) will be determined using first morning void (FMV) samples and will be calculated as the average of 2 FMV samples collected within 3 days prior to each visit. If 1 of the samples is missing, UA/C and UP/C from the single sample will be used.

For individual participants, the total duration of participation from the screening visit to the safety follow-up visit (Week 28) will be a maximum of 34 weeks.

### Sparsentan Treatment

Sparsentan will be administered daily as a 200-mg or 400-mg oral tablet. The starting dose will be 200 mg per day. The goal is to titrate from the initial dose of 200 mg (Day 1) to the target dose of 400 mg starting at Week 3.

### Endpoints

The incidence of treatment-emergent AEs (TEAEs), serious adverse events (SAEs), adverse events (AEs) leading to treatment discontinuation, and adverse event of interest (AEOIs), and changes from baseline in body weight, vital signs, physical examinations, peripheral edema, and clinical laboratory parameters, will be determined.

The change from baseline (Day 1) in UA/C at Week 24 will be determined to evaluate the effect of sparsentan on albuminuria in participants on chronic stable treatment with an SGLT2 inhibitor.

To evaluate the effect of sparsentan on proteinuria variables, estimated glomerular filtration rate (eGFR), and blood pressure (BP), in participants with IgAN on SGLT2 inhibitor medication over the duration of the study, the following will be determined: achievement of UA/C of <0.2 g/g at Week 24; achievement of 30% and 50% reduction from baseline in UA/C at Week 24; and the change from baseline in UA/C, UP/C, eGFR, and BP at each visit.

### Inclusion and Exclusion Criteria

Inclusion criteria will include:

Participant aged≥18 years at the time of signing the informed consent.

Participant has biopsy-proven IgAN. The biopsy may have been performed at any time in the past.

Participant has a UA/C≥0.3 g/g at screening.

Participant has an eGFR value of ≥25 mL/min/1.73 m2 at screening.

The participant has been on a stable dose of an SGLT2 inhibitor for at least 12 weeks prior to screening.

Participant has been on a stable dose of ACEI and/or ARB therapy for at least 12 weeks prior to screening that is: the participant's maximum tolerated dose (MTD), and at least one half of the maximum labeled dose (MLD).

Exclusion criteria will include:

Participant has IgAN secondary to another condition or immunoglobulin A (IgA) vasculitis.
Participant has undergone any organ transplant, with the exception of corneal transplants.
Participant has a documented history of heart failure, clinically significant cardiovascular or liver disease.
Participant has been taking high dose (defined as >10 mg/day prednisone) or other any systemic immunosuppressive medications within 12 weeks of prior to screening.

What is claimed is:

1. A composition for use in treating a kidney disease or disorder in a subject in need thereof, wherein the composition comprises sparsentan and the subject is also administered a SGLT2 inhibitor.

2. A composition for use in treating a proteinuric disease in a subject in need thereof, wherein the composition comprises sparsentan and the subject is also administered a SGLT2 inhibitor.

3. A composition for use in reducing proteinuria in a subject in need thereof, wherein the composition comprises sparsentan and the subject is also administered a SGLT2 inhibitor.

4. A composition for use in prolonging the time to end stage renal disease in a subject having a kidney disease or disorder, wherein the composition comprises sparsentan and the subject is also administered a SGLT2 inhibitor.

5. A composition for use in reducing a risk of end stage renal disease in a subject having a kidney disease or disorder, wherein the composition comprises sparsentan and the subject is also administered a SGLT2 inhibitor.

6. A composition for use in inducing complete remission in a subject having a kidney disease or disorder, wherein the composition comprises sparsentan and the subject is also administered a SGLT2 inhibitor.

7. The composition for use according to claim 6, wherein urinary protein to creatinine ratio (UP/C) is reduced to less than or equal to 0.3 g/g following administration of sparsentan and the SGLT2 inhibitor.

8. A composition for use in reducing the rate of decline in estimated glomerular filtration rate (eGFR) in a subject having a kidney disease or disorder, wherein the composition comprises sparsentan and the subject is also administered a SGLT2 inhibitor.

9. A method of treating a kidney disease or disorder in a subject in need thereof, the method comprising administering to said subject (i) sparsentan, and (ii) a SGLT2 inhibitor.

10. A method of treating a proteinuric disease in a subject in need thereof, the method comprising administering to said subject (i) sparsentan, and (ii) a SGLT2 inhibitor.

11. A method of reducing proteinuria in a subject in need thereof, the method comprising administering to said subject (i) sparsentan, and (ii) a SGLT2 inhibitor.

12. A method of prolonging the time to end stage renal disease in a subject having a kidney disease or disorder, the method comprising administering to said subject (i) sparsentan, and (ii) a SGLT2 inhibitor.

13. A method for reducing a risk of end stage renal disease in a subject having a kidney disease or disorder, the method comprising administering to said subject (i) sparsentan, and (ii) a SGLT2 inhibitor.

14. A method of inducing complete remission in a subject having a kidney disease or disorder, the method comprising administering to said subject (i) sparsentan, and (ii) a SGLT2 inhibitor.

15. The method according to claim 14, wherein urinary protein to creatinine ratio (UP/C) is reduced to less than or equal to 0.3 g/g following administration.

16. A method of reducing the rate of decline in estimated glomerular filtration rate (eGFR) in a subject having a kidney disease or disorder, the method comprising administering to said subject (i) sparsentan, and (ii) a SGLT2 inhibitor.

17. The composition for use or method according to any one of claims 1-16, wherein said SGLT2 inhibitor is dapagliflozin, empagliflozin, canagliflozin, ertugliflozin, ipragliflozin, tofogliflozin, or luseogliflozin, or a pharmaceutically acceptable salt, solvate, complex, or salts of solvates thereof.

18. The composition for use or method according to any one of claims 1-17, wherein the amount of sparsentan administered to said subject is from 50 mg/day to 1000 mg/day.

19. The composition for use or method according to any one of claims 1-18, wherein said sparsentan is administered orally.

20. The composition for use or method according to claim 19, wherein said sparsentan is administered with or without food.

21. The composition for use or method according to any one of claims 1-20, wherein the amount of said SGLT2 inhibitor administered to said subject is from 1 mg/day to 1000 mg/day.

22. The composition for use or method according to any one of claims 1-21, wherein said SGLT2 inhibitor is administered orally.

23. The composition for use or method according to claim 22, wherein said SGLT2 inhibitor is administered with or without food.

24. The composition for use or method according to any one of claims 1-23, wherein said kidney disease or disorder or proteinuric disease is focal segmental glomerulosclerosis (FSGS), minimal change disease (MCD), immunoglobulin A nephropathy (IgAN), immunoglobulin A-associated vasculitis (IgAV), or Alport syndrome.

25. The composition for use or method according to any one of claims 1-24, wherein said subject does not have severe hepatic impairment.

26. The composition for use or method according to any one of claims 1-25, wherein said subject does not have severe renal impairment.

27. The composition for use or method according to any one of claims 1-26, wherein said kidney disease or disorder is immunoglobulin A nephropathy (IgAN).

28. The composition for use or method according to claim 27, wherein said subject (i) has a baseline eGFR greater than or equal to 30 mL/min/1.73 m² and (ii) has a baseline UP/C greater than 1.0 g/g.

29. The composition for use or method according to claim 27, wherein said subject (i) has a baseline eGFR greater than or equal to 25 mL/min/1.73 m² and (ii) has a baseline urine albumin-to-creatinine ratio (UA/C) greater than 0.3 g/g.

30. The composition for use or method according to any one of claims 27-29, wherein said subject has a UP/C of less than or equal to 1.0 g/g following 36 weeks of treatment.

31. The composition for use or method according to any one of claims 27-30, wherein said subject has a UA/C of less than 0.2 g/g following 24 weeks of treatment.

32. The composition for use or method according to any one of claims 27-31, wherein said subject's UA/C is reduced by 30% to 50% from baseline UA/C following 24 weeks of treatment.

33. The composition for use or method according to any one of claims 1-26, wherein said kidney disease or disorder is focal segmental glomerulosclerosis (FSGS).

34. The composition for use or method according to claim 33, wherein said subject (i) has a baseline estimated glomerular filtration rate (eGFR) greater than or equal to 30 mL/min/1.73 m² and (ii) has a baseline UP/C greater than 1.5 g/g.

35. The composition for use or method according to claim 33 or claim 34, wherein the FSGS is primary FSGS.

36. The composition for use or method according to claim 35, wherein said subject has a UP/C of less than or equal to 1.5 g/g following 36 weeks of treatment.

37. The composition for use or method according to claim 35 or claim 36, wherein said subject's UP/C is reduced by more than 40% from the baseline UP/C following 36 weeks of treatment.

38. The composition for use or method according to any one of claims 1-26, wherein said kidney disease or disorder is Alport syndrome.

39. Sparsentan for use in the method according to any one of claims 9-38, wherein the subject is also administered a SGLT2 inhibitor according to any one of claims 9-38.

40. Use of sparsentan and a SGLT2 inhibitor in the manufacture of a medicament for use in the method according to any one of claims 9-38.

41. Use of sparsentan in the manufacture of a first medicament, and use of a SGLT2 inhibitor in the manufacture of a second medicament, wherein the first medicament and the second medicament are for use in the method according to any one of claims 9-38.
